(19) United States
(12) Patent Application Publication
(10) Pub. No.: US 2026/0098807 A1
(43) Pub. Date: Apr. 9, 2026

(54) METHODS FOR DETECTING PERFLUOROOCTANE SULFONIC ACID WITH PERYLENE-DIIMIDE-BASED CATIONIC FLUOROPHORES

(71) Applicants: University of Utah Research Foundation, Salt Lake City, UT (US); Gentex Corporation, Zeeland, MI (US)

(72) Inventors: Ling Zang, Salt Lake City, UT (US); Rana Dalapati, Salt Lake City, UT (US); Saravanakumar Manickam, Salt Lake City, UT (US)

(21) Appl. No.: 19/352,133

(22) Filed: Oct. 7, 2025

Related U.S. Application Data
(60) Provisional application No. 63/704,927, filed on Oct. 8, 2024.

(51) Int. Cl. G01N 21/64; G01N 33/18

(52) U.S. Cl. CPC G01N 21/643; G01N 33/18; G01N 2021/6439

(57) ABSTRACT

A method for detecting perfluorooctane sulfonic acid (PFOS) in an aqueous sample, the method including exciting the aqueous sample alone at a wavelength in a range of approximately 480 to 500 nm, contacting the aqueous sample with a detection reagent comprising a perylene-diimide-based cationic fluorophore, exciting the detection reagent and aqueous sample combination at a wavelength in a range of approximately 480 to 500 nm, and further quantifying a change in a fluorescence intensity of the detection reagent while in contact with the sample relative to the fluorescence intensity of the detection reagent prior to being contacted with the sample.

### CROSS-REFERENCE TO RELATED APPLICATION

This application claims priority to and the benefit under 35 U.S.C. § 119(e) of U.S. Provisional Patent Application No. 63/704,927, filed on Oct. 8, 2024, entitled METHODS FOR DETECTING PERFLUOROOCTANE SULFONIC ACID WITH PERYLENE-DIIMIDE-BASED CATIONIC FLUOROPHORES, the entire disclosure of which is hereby incorporated herein by reference.

### FIELD OF THE DISCLOSURE

The present disclosure generally relates to methods for detecting perfluorooctane sulfonic acid (PFOS) in a sample, and more particularly to methods for detecting PFOS with perylene-diimide-based cationic fluorophores.

### BACKGROUND

Per- and poly-fluoroalkyl substances (PFAS), also known “forever chemicals,” are an emerging class of pollutants widely present in surface/ground waters and soils. These compounds have been used for over 60 years in hundreds of industrial applications and consumer products [e.g., carpet, apparel, upholstery, cookware, food wrappers, and aqueous fire-fighting foams (AFFFs)]. More than 9,000 PFAS have been identified, with the perfluorooctane sulfonic acid (PFOS) and perfluorooctanoic acid (PFOA) being the most used and widely studied. The term “forever chemicals” reflects the high chemical stability of PFAS, which renders them highly resistant to hydrolysis, biodegradation, metabolism, photolysis (sunlight), and other degradation processes. The U.S. and European countries have begun to ban the manufacturing and use of many PFAS, but efforts to remediate the global spread of PFAS will likely take several decades.

Currently, the official health advisory level set by the U.S. Environment Protection Agency for PFOA and PFOS in drinking water is 70 ppt, a level that may be markedly lowered to 0.004 ppt for PFOA and 0.02 ppt for PFOS according to an advisory set on Jun. 15, 2022, pending the results of ongoing health-related studies. According to recent speculation, implementing new guidelines and regulations may make water everywhere, including fresh rainwater, being labeled as contaminated and unfit for consumption and use. To this point, new methods will be required that are highly sensitive for the detection of PFAS. Moreover, these methods may also need to be highly selective for specific PFAS, such as PFOA and PFOS, so that appropriate remediation techniques can be used and the sources of contamination can be ascertained. Improved detection methods also may aid efforts to gauge the range and scope of the geographic distribution of PFAS contamination and assist in monitoring the efficiency of treatment.

Current PFAS detection methods are poorly suited for field deployment due in part to requirements such as derivatization prior to analysis, lengthy sample preparation, cost, and instrument maintenance. Predominant methods include mass spectrometry (MS)-based ex-situ laboratory techniques. Commonly used modes include analytical scale extraction and subsequent analysis by liquid chromatography tandem mass spectrometry (LC-MS/MS) and total oxidizable precursor (TOP). An alternative approach to MS involves the analysis of total fluorine by particle induced gamma ray emission (PIGE) spectroscopy. Again, these methods are poorly suited to use in the field. Moreover, the limits of detection (LOD) for the most recent EPA-validated methods is 0.3 ppt for PFOS and PFOA, which falls short of the advisory level by nearly one hundred-fold.

Additionally, prior probes and sorbents for PFAS capture and detection have exhibited poor uptake, slow kinetics, and/or poor selectivity. Some prior probes have been based on a general electronic affinity for electron rich hydrophobic, groups (e.g., perfluoroalkyl or similar species). While prior sensors were able to detect PFOS and PFOA in simple matrices (DI water and drinking water), the nonspecific binding limited both selectivity and sensitivity in practical matrices, due to the inability to screen out interferences with similar electronic properties and hydrophobicity. For at least these reasons, there is a need for sensitive and selective compounds that can detect and quantify various PFAS, such as PFOS and PFOA.

### SUMMARY OF THE DISCLOSURE

According to one aspect of the present disclosure, a method for detecting perfluorooctane sulfonic acid (PFOS) in a sample, includes contacting the sample with a detection reagent comprising a compound of formula (I), or a salt thereof:

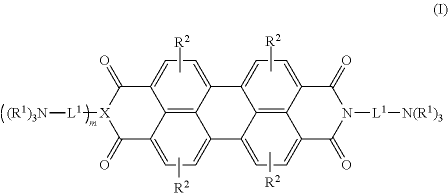

X is either N or O, when X is N, m is 1 and wherein when X is O, m is O, L¹, at each occurrence, is independently C₁₋₆alkylene or C₁₋₃alkylene-C═C—C₁₋₃alkylene, R¹, at each occurrence, is independently hydrogen, C₁₋₆alkyl, or C₁₋₆alkylene-N(R^{y})ₙ, n=1-3, R², at each occurrence, is independently hydrogen, C₁₋₆alkyl, halogen, or —O(R^{y}), and R^{y}, at each occurrence, is independently hydrogen or C₁₋₄alkyl. The method further includes quantifying a change in a fluorescence intensity of the detection agent while in contact with the sample relative to the fluorescence intensity of the detection agent prior to being contacted with the sample.

According to another aspect of the present disclosure, a method for detecting perfluorooctane sulfonic acid (PFOS) in a sample, includes contacting the sample with a detection reagent comprising a compound of formula (II), or a salt thereof:

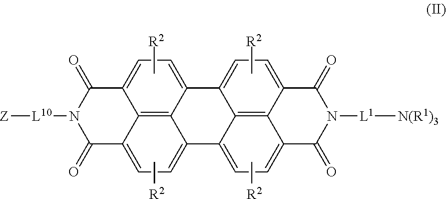

L¹ is C₁₋₆alkylene or C₁₋₃alkylene-C═C—C₁₋₃alkylene, R¹, at each occurrence, is independently hydrogen, C₁₋₆alkyl, or C₁₋₆alkylene-N(R^{x})ₙ, n=1-3, R^{y}, at each occurrence, is independently hydrogen, C₁₋₆alkyl, halogen, or —O(R^{y}), and R^{X}, at each occurrence, is independently hydrogen or C₁₋₄alkyl, L¹⁰ is a linker, Z is a solid support. The method further includes quantifying a change in a fluorescence intensity of the detection agent while in contact with the sample relative to the fluorescence intensity of the detection agent prior to being contacted with the sample.

According to yet another aspect of the present disclosure, a method for detecting perfluorooctane sulfonic acid (PFOS) in an aqueous sample, includes exciting the aqueous sample alone at a wavelength in a range of approximately 480 to 500 nm, contacting the aqueous sample with a detection reagent comprising a perylene-diimide-based cationic fluorophore, exciting the detection reagent and aqueous sample combination at a wavelength in a range of approximately 480 to 500 nm, and quantifying a change in a fluorescence intensity of the detection reagent while in contact with the sample relative to the fluorescence intensity of the detection reagent prior to being contacted with the sample.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1A is graph illustrating a series of fluorescence emission spectra of PDI-6+ in the presence of varying concentrations of PFOS;

FIG. 1B is a linear regression plot of fluorescence intensity of PDI-6+ in the presence of varying amounts of PFOS;

FIG. 1C is a linear regression plot of fluorescence intensity of PDI-6+ as a function of a concentration of PFOS;

FIG. 2 is a Stern-Volmer plot of Iₒ/I for PDI-6+ as a function of a concentration of PFOS;

FIG. 3A is a series of UV-vis absorption spectra of PDI-6+ in the presence of varying concentrations of PFOS;

FIG. 3B is a linear regression plot of an absorbance of PDI-6+ as a function of a concentration of PFOS;

FIG. 4A is a bar chart showing the quenching efficiency of PDI-6+ in the presence of various other analytes relative to the intensity of the fluorescence intensity of PDI-6+ in the absence of the analyte;

FIG. 4B is a bar chart showing interference testing that compares the ability of PFOS to quench fluorescence emission of PDI-6+, thus showing the selectivity of PDI-6+ towards PFOS;

FIG. 5 is a Job plot showing the stoichiometry of the complex between PDI-6+ and PFOS;

FIG. 6 is a plot of fluorescence emission intensity PDI-6+ in the absence of PFOS and in the presence of PFOS as a function of time;

FIG. 7 is a plot of fluorescence emission intensity of PDI-6+ in the absence of PFOS and in the presence PFOS as a function of temperature;

FIG. 8A is a plot of fluorescence emission intensity of PDI-6+ in the absence of PFOS and in the presence of PFOS as a function of pH;

FIG. 8B is a series of fluorescence emission spectra of PDI-6+ at varying pH levels;

FIG. 8C is a series of fluorescence emission spectra of PDI-6+ in the presence of PFOS at varying pH levels;

FIG. 9A is a plot of fluorescence emission intensity of PDI-6+ in the absence of PFOS and in the presence of as a function of ionic strength;

FIG. 9B is a plot of fluorescence emission intensity of PDI-6+ in the absence of PFOS and in the presence of PFOS as a function of ionic strength;

FIG. 9C is a plot of fluorescence emission intensity of PDI-6+ in the absence of PFOS and in the presence of PFOS as a function of ionic strength;

FIG. 10A is a series of images (in color) showing a fluorescence of filter paper strips that have been immersed in PDI-6+ solution and contacted with solutions having varying concentrations of PFOS;

FIG. 10B is a series of images (in color) showing a fluorescence of filter paper strips that have been immersed in PDI-6+ solution and contacted with solutions having varying concentrations of PFOS;

FIG. 11A is an image of a series of vials containing solutions of PDI-6+ with varying concentrations of PFOS;

FIG. 11B is a plot of RGB values from the samples of FIG. 11A;

FIG. 12 is a series of fluorescence emission spectra of PDI-2+ in water in the presence of varying concentrations of PFOS;

FIG. 13 is a linear regression plot of fluorescence intensity of PDI-2+ in water as a function of the concentration of PFOS;

FIG. 14 is a schematic illustration of a proposed mechanism of fluorescence quenching;

FIG. 15 is a bar chart illustrating quenching efficiency of PDI-2+ with various analytes;

FIG. 16 is a bar chart illustrating interference testing of PFOS to quench PDI-2+;

FIG. 17 is a series of absorption spectra of PDI-2+ in the presence of varying concentrations of PFOS;

FIG. 18 is a Job's plot illustrating stoichiometry of the complex between PDI-2+ and PFOS;

FIG. 19 is a series of fluorescence emission spectra of 5 μSG-PDI-3+ in water in the presence of varying concentrations of PFOS;

FIG. 20 is a Stern-Volmer plot of Iₒ/I for 5 μSG-PDI-3+ as a function of the concentration of PFOS (0-2.5×10⁻⁵ M);

FIG. 21 is a series of fluorescence emission spectra of 5 μSG-PDI-1+ in water in the presence of varying concentrations of PFOS; and

FIG. 22 is a Stern-Volmer plot of Iₒ/I for 5 μSG-PDI-1+ as a function of the concentration of PFOS.

### DETAILED DESCRIPTION

The present illustrated embodiments reside primarily in combinations of method steps and apparatus components related to fluorescent sensors for detection of perfluorooctane sulfonic acid in a sample. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

It is to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

The present disclosure relates to aqueous detection methods for detecting perfluorooctane sulfonic acid (PFOS) with the perylene-diimide (PDI)-based cationic fluorophores. In some implementations, the PDI molecule is modified with cationic side groups at both imide positions. These methods of detection are both highly sensitive (having limits of detection of between about 1 and about 50 ppb) and highly selective, enabling sensitive detection of PFOS even in the presence of other PFAS chemicals. In some examples, the PDI-based fluorophore is specifically a water-soluble cationic perylene diimide derivative such as PDI-2+, PDI-6+, and PDI-CI-2+, which are based on a perylene diimide (PDI) structure modified with two or six quaternary trimethylammonium cationic side groups. Under optimal conditions, the limit of detection (LOD) for PDI-2+ can reach as low as 7 nM (or 3.5 ppb).

Definitions of specific functional groups and chemical terms are described in more detail below.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkenyl,” as used herein, means a straight or branched, hydrocarbon chain containing at least one carbon-carbon double bond.

The term “alkylene” as used herein, refers to a bivalent saturated aliphatic radical, such as ethylene (—CH₂—CH₂—), which bridges two other groups in a molecule.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRxRy, wherein Rx and Ry may independently be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRx-, wherein Rx may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and may have from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms “cycloalkylene” and “heterocyclylene” refer to divalent groups derived from the base ring, i.e., cycloalkane, heterocycle. For illustration, an example cycloalkylene may be cyclohexene or

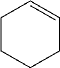

and a heterocyclylene may be

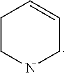

Cycloalkylene and heterocyclylene include a geminal divalent group such as 1,1-C₃₋₆cycloalkylene. A further example is 1,1-cyclopropylene.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

“Fluorescence” as used herein is a cyclical process where a luminescence is generated by certain molecules in which the molecular absorption of a photon triggers the emission of another photon with a longer wavelength. Certain molecules are capable of being excited, via absorption of light energy, to a higher energy state, also called an excited state. The energy of this short-lived excited state decays (or decreases), resulting in the emission of light energy. The emission of light via this process is “fluorescence.” Molecules that emit light in this manner are said to “fluoresce” and are generally referred to as “fluorophores” or “fluorescent dyes.”

A “fluorophore” or “fluorescent dye,” as used herein, is a molecule that is capable of fluorescing. In its ground state, the fluorophore molecule is in a relatively low-energy, stable configuration, and the molecule does not fluoresce. When light from an external source of one or more particular wavelengths contacts a fluorophore, the fluorophore can absorb the light energy. If the fluorophore absorbs sufficient energy (e.g., through a process of exciting), the fluorophore is excited to an excited state (high energy); this process is known as excitation. There may be multiple excited states or high energy levels that a fluorophore can attain, depending on the wavelength and energy of the external light source. Since a fluorophore is unstable at high-energy configurations, the fluorophore eventually decays to the lowest-energy excited state, which is semi-stable. The excited lifetime (the length of time that a fluorophore is an excited state) is very short; the fluorophore then decays from the semi-stable excited state back to the ground state, and at least a portion of the excess energy released by this decay may be emitted as light. The emitted light is of a lower energy, and a longer wavelength, than the absorbed light, and thus the color of the light that is emitted is different from the color of the light that has been absorbed. Upon reaching the ground state, a fluorophore can again absorb light energy to enter an excited state.

A fluorophore or fluorescent dye absorbs light over a range of wavelengths and every dye has a characteristic range of excitation wavelengths. This range of excitation wavelengths is referred to as the fluorescence “excitation spectrum,” “absorption spectrum” and/or “absorbance spectrum” and reflects the range of possible excited states that the dye can achieve. Certain wavelengths within this range are more effective for excitation than other wavelengths. A fluorophore is excited most efficiently by light of a particular wavelength. This wavelength is the excitation maximum for the fluorophore. As used herein “excitation maximum” refers to the specific wavelength for each fluorescent dye that most effectively induces fluorescence. Less efficient excitation can occur at wavelengths near the excitation maximum; however, the intensity of the emitted fluorescence is reduced. Although illumination at the excitation maximum of the fluorophore produces the greatest fluorescence output, illumination at lower or higher wavelengths affects only the intensity of the emitted light; the range and overall shape of the emission profile are unchanged.

As used herein, “excitation” refers to the process where a photon of energy supplied by an external source, such as a laser or a lamp, is absorbed by the fluorophore creating an excited electronic singlet state (S₁′) from a S₀ ground state. The excited state exists for a finite time during which the fluorophore undergoes conformational changes which may include, but are not limited to: changes to an electron density redistribution of the fluorophore, vibrational relaxation, and/or molecular geometry changes. The fluorophore is also subject to a multitude of possible interactions with its molecular environment. If the fluorophore is dissolved within a solvent, the fluorophore may undergo conformational changes to better interact with the local environment due to new solvation effects and/or new non-covalent interactions with the surrounding solvent molecules. These processes have two important consequences. One of these consequences is that the energy of S₁′ is partially dissipated, yielding a relaxed singlet excited state (S₁) from which fluorescence emission originates. Molecules in an excited state (S₁′) can relax by various competing pathways. They can undergo ‘non-radiative relaxation’ in which the excitation energy is dissipated as heat (vibrations) to the solvent. Excited organic molecules can also relax via conversion to a triplet state which may subsequently relax via phosphorescence or by a secondary non-radiative relaxation step. The term “relax” as used herein refers to the energy loss of an excited molecule. Not all the molecules initially excited by absorption return to the ground state (S₀) by fluorescence emission. Relaxation of an S₁′ state can also occur through interaction with a second molecule through fluorescence quenching. Other processes, such as, but not limited to, collisional quenching or fluorescence resonance energy transfer (FRET), may also depopulate S₁.

FRET is a radiationless process in which energy is transferred from an excited donor molecule to an acceptor molecule. Radiationless energy transfer is the quantum-mechanical process by which the energy of the excited state of one fluorophore is transferred without actual photon emission to a second fluorophore or molecule. Briefly, a fluorophore absorbs light energy at a characteristic wavelength. The first fluorophore is generally termed the donor (“D”) and may have an excited state of higher energy than that of the second fluorophore, termed the acceptor (“A”).

An essential feature of FRET is that the emission spectrum of the donor overlap with the excitation spectrum of the acceptor, and that the donor and acceptor be sufficiently close. In addition, the distance between “D” and “A” must be sufficiently small to allow the radiationless transfer of energy between the fluorophores. Because the rate of energy transfer is inversely proportional to the sixth power of the distance between the donor and acceptor, the energy transfer efficiency is extremely sensitive to distance changes. Energy transfer is said to occur with detectable efficiency in the 1-10 nm distance range but is typically detected at about 4-6 nm for optimal results. The energy transfer may additionally or alternatively occur when the donor and acceptor are attached or tethered to the same molecule or molecular structure. The distance range over which radiationless energy transfer is effective depends on many other factors as well, including the fluorescence quantum efficiency of the donor, the extinction coefficient of the acceptor, the degree of overlap of their respective spectra, the refractive index of the medium, and the relative orientation of the transition moments of the two fluorophores.

As used herein, the term “quencher” means a substance, which reduces or quenches the emission of fluorescence from a fluorophore. As used herein, “fluorescence quenching” may be achieved by any mechanism, typically by FRET between a fluorophore and a non-fluorescent quenching moiety or by collisional (i.e., contact) quenching.

Fluorophore molecules, when excited, emit over a range of wavelengths. This range of wavelengths is referred to as the fluorescence “emission spectrum.” There is a spectrum of energy changes associated with these emission events. The emission maximum is the wavelength where the population of molecules fluoresces most intensely. The emission maximum for a given fluorophore is always at a longer wavelength (lower energy) than the excitation maximum. This difference between the excitation and emission maxima is called the Stokes shift. The magnitude of the Stokes shift is determined by the electronic structure of the fluorophore and is characteristic of the fluorophore molecule. The Stokes shift occurs because some of the energy of the excited fluorophore is lost through molecular vibrations that occur during the brief lifetime of the molecule's excited state, which is dissipated as heat to surrounding solvent molecules as they collide with the excited fluorophore. Remaining energy that is emitted as light fluorescence is thus less than the amount of energy required for excitation.

Fluorescence requires a source of excitation energy. There are many light source options for fluorescence. Selecting the appropriate light source, and filters for both excitation and emission, can increase the sensitivity of signal detection. Several types of light sources are used to excite fluorescent dyes. The most common sources used are broadband sources, such as, for example, mercury-arc and tungsten-halogen lamps. These lamps produce white light that has peaks of varying intensity across the spectrum. When using broadband white light sources, it is necessary to filter the desired wavelengths needed for excitation; this is most often done using optical filters. Optical filters selectively allow light of certain wavelengths to pass while blocking out undesirable wavelengths. A bandpass excitation filter transmits a narrow range of wavelengths and may be used for selective excitation according to methods of the present disclosure.

Laser excitation sources provide wavelength peaks that are well-defined, selective, and of high intensity allowing more selective illumination of the sample. The best performance is achieved when the dye's peak excitation wavelength is close to the wavelength of the laser. Several lasers commonly used include, for example, the compact violet 405 nm laser, 488 nm blue-green argon-ion laser, 543 nm helium-neon green laser, and 633 nm helium-neon red laser. Mixed-gas lasers such as, for example, the krypton-argon laser, can output multiple laser lines which may require optical filters to achieve selective excitation. High-output light-emitting diodes (LEDs) provide selective wavelengths, low cost and energy consumption, and long lifetimes. Single-color LEDs are ideal for low-cost instrumentation where they can be combined with simple long-pass filters that block the LED excitation and allow the transmission of the dye signal. However, the range of wavelengths emitted from each LED is still relatively broad and may also require the use of a filter to narrow the bandwidth.

Filters are important for selecting excitation wavelengths and for isolating the fluorescence emission emanating from the dye of interest, such as the detection reagent according to the present disclosure. Stray light arising from sources other than the emitting fluorophores (for example, from the excitation source) interferes with the detection of the fluorescence emission. Stray light, therefore, must be contained to ensure only the fluorescence of the sample registers with the instrument's light-sensitive detectors. When a single fluorophore is used, a long pass emission filter that selectively blocks out the excitation light to reduce background noise may be used to maximize the signal collected. If multiple fluorophores are used in the sample, a band pass emission filter can be used to isolate the emission from each dye.

Generally, the methods of the present disclosure include the use of PDI-based fluorophores to selectively detect and/or quantify perfluorooctane sulfonic acid (PFOS) in a sample. These methods include contacting a sample with the PDI-based fluorophores disclosed herein. If PFOS is present in the sample, then the PFOS interacts with the PDI-based fluorophores in a manner that induces a change in ability of the PDI-based fluorophore to fluoresce at particular emission wavelength(s). Changes in fluorescence intensity of the PDI-based fluorophores when they come into contact with a sample can thus be used to detect and/or quantify the amount of PFOS in the sample. The PDI-based fluorophores disclosed herein can, therefore, be used as detection reagents.

The PDI-based fluorophores of the present disclosure are configured to selectively interact with PFOS in a highly sensitive manner that significantly changes their emission intensities at a particular emission wavelength (i.e., the PDI-based fluorophores are used as detection reagents). According to the present disclosure, the PDI-based fluorophores do not interact with other PFAS chemicals in the same manner, thereby enabling these fluorophores to selectively detect PFOS specifically in samples containing PFOS with, or without, other PFAS chemicals. Further, the PDI-based fluorophores do not interact with other PFAS, like GenX, structurally similar detergents, and inorganic salts typically found in water.

According to the method of the present disclosure, the interaction between the PDI-based fluorophores of the present disclosure and PFOS causes a decrease in the fluorescence of the fluorophore at one or more emission wavelengths (e.g., PFOS functions as a fluorescence quencher to the PDI-based fluorophores disclosed herein). These changes in fluorescence of the fluorophore allow for use of the fluorophore to detect and/or quantify PFOS in a sample with limits of detection between about 1 and about 50 parts per billion (ppb). To detect and/or quantify PFOS in a sample at these limits of detection, the detection reagent (e.g., the fluorophore) may be excited at an excitation wavelength of between about 460 nm and about 510 nm, between about 470 nm and about 500 nm, between about 480 nm and about 490 nm (both in the presence and in the absence of the sample), and the fluorescence intensity may then be quantified at an emission wavelength of between about 520 nm and about 620 nm, between about 530 nm and about 610 nm, between about 540 nm and about 600 nm. It has been found that the formation of a supramolecular complex between PFOS and the cationic fluorophores, facilitated by the synergistic interplay of electrostatic, hydrophobic, π-π stacking interactions, and/or other supramolecular interactions enables a rapid fluorometric sensing response for the detection of PFOS in aqueous systems. Without wishing to be bound by theory, the electrostatic interactions may occur with positively charged amine groups on the fluorophore and with the sulfonate group of the PFOS, and the hydrophobic interactions may occur between the fluoroalkyl chains between different PFOS molecules. Likewise, the π-π stacking interactions may occur between the aromatic ring structure between different fluorophore molecules causing them to stack. Remarkably, the detection limit for PFOS using methods of the present disclosure was found to be as low as 7 nM (3.7 ppb) for PDI-2+ and 14.7 nM (7.9 ppb) for PDI-6+, showcasing the high sensitivity of the sensor for PFOS detection.

In one aspect of the present disclosure, a method for detecting perfluorooctane sulfonic acid (PFOS) in a sample includes contacting the sample with a detection reagent including a compound of formula (I), or a salt thereof:

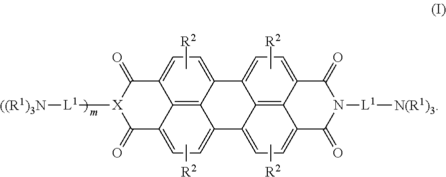

wherein X is either N or O, wherein when X is N, m is 1 and wherein when X is O, m is 0, L¹, at each occurrence, is independently C₁₋₆alkylene or C₁₋₃alkylene-C═C—C₁₋₃alkylene, R¹, at each occurrence, is independently hydrogen, C₁₋₆alkyl, or C₁₋₆alkylene-N(R^{Y})ₙ, wherein n=1-3, R², at each occurrence, is independently hydrogen, C₁₋₆alkyl, halogen, or —O(R^{Y}), and R^{Y}, at each occurrence, is independently hydrogen or C₁₋₄alkyl.

The method includes quantifying a change in a fluorescence intensity of the detection reagent while in contact with the sample relative to the fluorescence intensity of the detection agent prior to being contacted with the sample. In some implementations R², at each occurrence, is independently hydrogen.

In some implementations, the compound of formula (I), or a salt thereof, is the compound of formula (Ia), or a salt thereof:

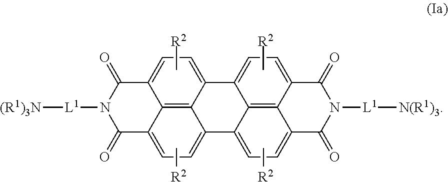

In another example, the compound of formula (Ia), or a salt thereof, is, more specifically, the compound of formula (Ib), or a salt thereof:

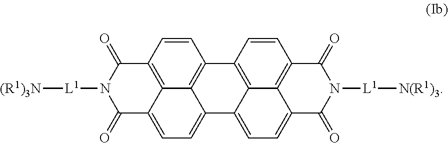

In some implementations, the compound of formula (I), or a salt thereof, is the compound of formula (Ic), or a salt thereof:

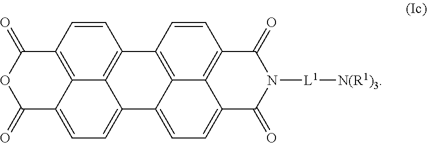

In yet another example, the compound of formula (Ic), or a salt thereof, is, more specifically, the compound of formula (Id), or a salt thereof:

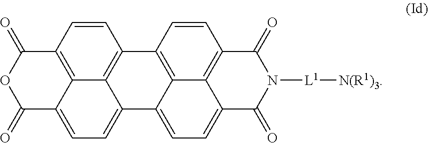

In some aspects of the present disclosure, L¹ may be C₁₋₆alkylene, R¹ may be C₁₋₆alkyl or C₁₋₆alkylene-N(R^{Y})ₙ, and n may be equal to 3, R^{Y} may be C₁₋₄alkyl. Additionally, the compound of formula (I), or a salt thereof, may be selected from the group consisting of:

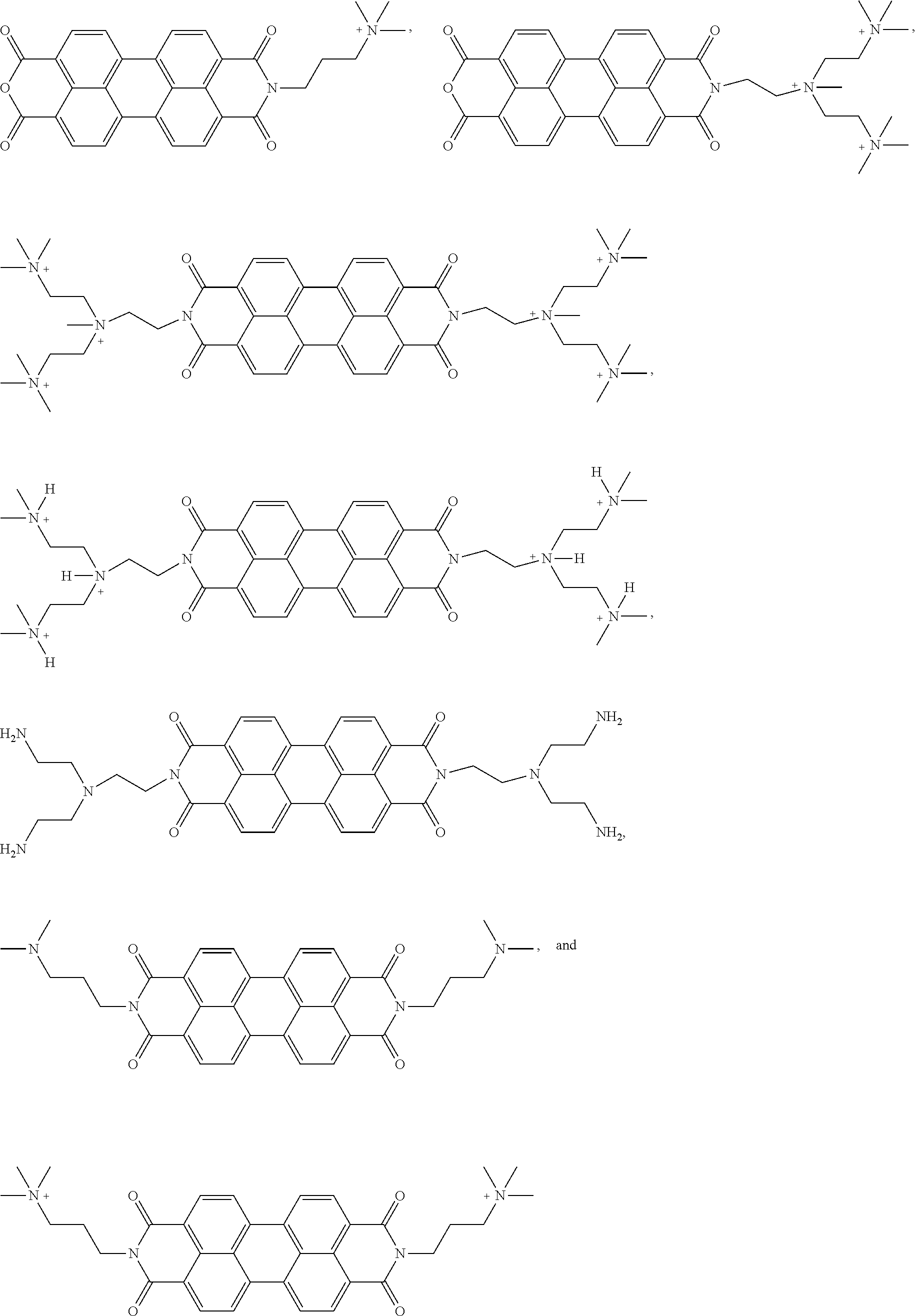

The PDI-based fluorophores of the present disclosure may be covalently attached or non-covalently adhered to a surface of a solid support. In some examples, the solid support may include a nanoparticle, nanofiber, microparticle or microfiber having average sizes ranging from about 1 nm to about 500 μm in diameter. In specific examples, the average particle size may be from 10 μm to about 15 μm. The nanoparticles, nanofibers, microparticles or microfibers may be soluble or soluble in a particular solvent, may form colloidal suspensions and/or may adsorb liquids. The solid support may be a solid phase extraction membrane or “SPE membrane.” SPE membranes may include porous structures having pore sizes ranging from about 0.1 μm to about 10 μm. The SPE membrane may be housed in a filter disk and/or a filter column having diameters ranging from about 5 mm to about 100 mm and the solid support may be used to form a column for use in chromatographic separations.

The solid support and, therefore a surface thereof, may be formed of any suitable material, including, but not limited to: a cellulose, a metal oxide, a polymer, a resin, a silica, and combinations thereof. The suitable material may have sufficient porosity to be utilized as the solid support as described herein. The selection of a particular substrate generally depends on various criteria, including, but not limited to, chemical stability, photostability, the ability (or lack thereof) of the substrate to absorb excitation or emission light, ease of surface modification, cost and environmental toxicity (or lack thereof). Suitable celluloses may include, but are not limited to, a nitrocellulose and a cellulose acetate. Suitable metal oxides may include but are not limited to titania (TiO₂), zinc oxide (ZnO) and alumina (Al₂O₃). Suitable polymers may include, but are not limited to a polyacrylate, a polyacrylonitrile, a polycarbonate, a polyimide, a polymethyl methacrylate, a polypropylene, a polytetrafluoroethylene, and a polyvinylidene difluoride. For example, the polymer may include a polystyrenedivinylbenzene and a sulfonated polystyrenedivinylbenzene. Resins may include any suitable cation or anion exchange resin. Silicas may include, but are not limited to, fumed silica, precipitated silica and silicas produced through aerosol assisted self-assembly. Exemplary silicas may include, but are not limited to, C₈ and C₁₈ bonded silica.

As previously described, the PDI-based fluorophores of the present disclosure may be non-covalently adhered to the surface of a solid support. For example, the PDI-based fluorophores may be bound to the surface by a ligand that binds to the fluorophore via intermolecular interactions including, but not limited to, electrostatic interactions, π-effects interactions, hydrogen bonding, van der Waals forces, and/or hydrophobic interactions, and the like. However, the PDI-based fluorophores also may be covalently attached to the surface of the solid support through reactions with reactive functional groups that chemically react with the fluorophore to form a covalent bond. Upon reaction with a functional group, the fluorophore may then be coupled, tethered or linked to the solid support with a linker. In some aspects, any suitable chemistry used to couple compounds to solid supports may be used according to the present disclosure, provided the chemistry includes an appropriate functional group that can react with a reactive moiety on the fluorophore.

In some examples, the PDI-based fluorophores of the present disclosure may be coupled to the solid support by functionalizing the surface of the solid support with a primary amine functional group, and then reacting the primary amine with the compound of formula (Ic):

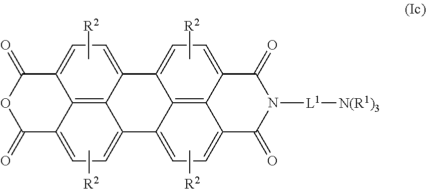

to form the compound of formula (II), or a salt thereof:

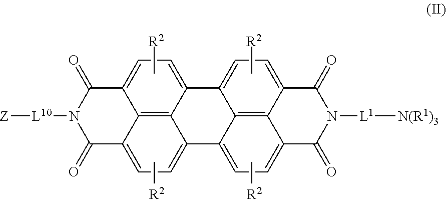

where Z is a solid support and L¹⁰ is a linker.

Solid supports that have been functionalized with primary amine functional groups are referred to herein as “amine-functionalized solid supports.” In some aspects of the present disclosure, amine-functionalized solid supports may include —C₁₋₁₀alkylene-NH₂ chemically coupled to the solid support, such that, upon reaction of the amine with the compound of Formula (Ic), the linker L¹⁰ includes the C₁₋₁₀alkylene. For example, silica may be amine-functionalized using 3-aminopropyl(trimethoxysilane) (APTMS) or 3-aminopropyl(trimethoxysilane) (APTES) according to various reactions, and, optionally, as shown in Scheme 2 of the examples, below. Reacting the compound of formula (Ic) with the APTES-functionalized or APTMS-functionalized silica forms the compound of formula (II) where L¹⁰ includes C₃alkylene.

Compounds of formula (II) may be used in various methods for detecting PFOS, according to the following examples.

In another aspect of the present disclosure, the method for detecting perfluorooctane sulfonic acid (PFOS) includes contacting the sample with a detection reagent including a compound of formula (II), or a salt thereof:

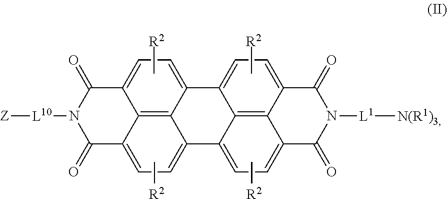

wherein L¹ is C₁₋₆alkylene or C₁₋₃alkylene-C═C—C₁₋₃alkylene, R¹, at each occurrence, is independently hydrogen, C₁₋₆alkyl, or C₁₋₆alkylene-N(R^{Y})ₙ, wherein n=1-3, R², at each occurrence, is independently hydrogen, C₁₋₆alkyl, halogen, or —O(R^{Y}), and R^{Y}, at each occurrence, is independently hydrogen or C₁₋₄alkyl, L¹⁰ is a linker, and Z is a solid support. In some examples, the solid support Z may be formed of a material including a silica, a polymer and a resin, and combinations thereof.

The method includes quantifying a change in a fluorescence intensity of the detection agent while in contact with the sample relative to the fluorescence intensity of the detection agent prior to being contacted with the sample.

In specific examples, R², at each occurrence, is hydrogen, L¹ is C₁₋₆alkylene and R¹ is C₁₋₆alkyl or C₁₋₆alkylene-N(R^{Y})ₙ. In further specific examples, n may be equal to three and R^{Y} may be C₁₋₄alkyl.

In specific examples, the compound of formula (II), or a salt thereof, is the reaction product of an amine-functionalized solid support and the compound of formula (Ic):

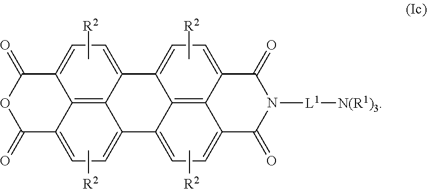

The amine-functionalized solid support compound of formula (Ic) may include —C₁₋₁₀alkylene-NH₂ chemically coupled to the solid support, where L¹⁰ includes C₁₋₁₀alkylene. Further, the amine-functionalized solid support may be amine-functionalized silica. In specific examples, the amine-functionalized silica is the product of a reaction between silica and either APTMS or APTES, where L¹⁰ includes C₃alkylene.

The PDI-based fluorophores of the present disclosure may exist as salts when not dissolved in a solvent. The salts may exist as ionic compounds with at least one counterion that stabilizes their ionic charge when in solution dissolved in a solvent. Examples of counterions include, but are not limited to, iodide, chloride, bromide, and combinations thereof. Examples of solvents include, but are not limited to, aqueous and organic solvents. In some examples, an aqueous solvent may also include various dissolved solutes. Organic solvents may include, but are not limited to, acetonitrile, ethanol, methanol, tetrahydrofuran, DMSO, and DMF. Additionally, the PDI-based fluorophores described herein each have slightly different absorption spectra and slightly different emission spectra when excited at particular excitation wavelengths. Peak absorption wavelengths for the PDI-based fluorophores range from about 480 nm to about 500 nm. When excited at peak absorption wavelengths, the PDI-based fluorophores may have peak emission wavelengths between about 520 nm and about 700 nm, and, more specifically, between about 540 nm and about 600 nm.

### EXAMPLES

### Example 1: Determination of Polyfluorooctane Sulfonic Acid (PFOS) with Fluorescent Probe PDI-6+

### Abbreviations

PTCDA is perylene tetracarboxylic dianhydride;
NMR is nuclear magnetic resonance;
TLC is thin-layer chromatography;
eq. or equiv. is equivalents;
min or min. is minute(s);
h or hr. is hour(s);
rt, RT, or r.t. is room temperature; and
sat'd or sat. is saturated.

### Exemplary Synthesis of Perylene Diimides (PDI)

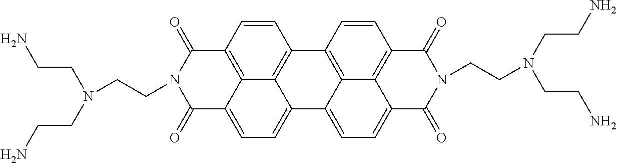 [SMILES: NCCN(CCN)CCN1C(=O)c2ccc3c4ccc5c6c(ccc(c7ccc(c2c37)C1=O)c64)C(=O)N(CCN(CCN)CCN)C5=O]

2,9-bis(2-(bis(2-aminoethyl)amino)ethyl)anthra[2,1,9-def:6,5,10-d′e′f′]diisoquinoline-1,3,8,10(2H,9H)-tetraone: PDA (0.2 g, 0.51 mmol) and tris-(2-aminoethyl)amine (3.85 mL, 25.4 mmol) were mixed. During the first 24 hours, the mixture was stirred at 100° C., then the temperature gradually increased to 170° C. over 4 hours. In a subsequent step, the mixture was cooled to room temperature and ethanol was added in a ratio of 1:3 to diethyl ether. A brown solid compound was obtained by suction filtration, washing with toluene and diethyl ether, and drying under vacuum in 87% yield.¹H-NMR (400 MHz, D₂SO₄): 8.77-8.65 (m, 8H), 6.28 (m, 4H), 4.53 (m, 4H), 3.53-3.40 (m, 16H). Exact mass calculated for C₃₆H₄₀N₈O₄: 648.32. Found: 649.31 (MH⁺ ion peak).

### Exemplary Synthesis of PDI-Based Fluorophores

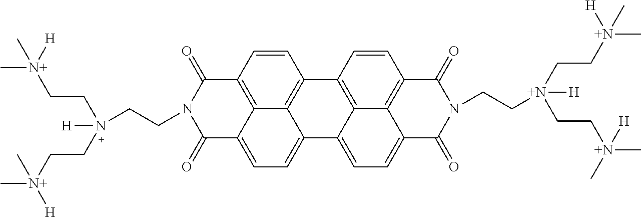

N¹,N^{1′}-((1,3,8,10-tetraoxo-1,3,8,10-tetrahydroanthra[2,1,9-def:6,5,10-d′e′f′]diisoquinoline-2,9-diyl)bis(ethane-2,1-diyl))bis(N1-(2-(dimethylammonio)ethyl)-N²,N²-dimethylethane-1,2-diaminium): 0.24 g of 2,9-bis(2-(bis(2-aminoethyl)amino)ethyl)anthra[2,1,9-def:6,5,10-d′e′f′]diisoquinoline-1,3,8,10(2H,9H)-tetraone, 1.2 mL of water, 1.4 mL of 97% formic acid, and 1.3 mL of 37% formaldehyde were mixed. The mixture was first stirred at room temperature for an hour and then heated at 120° C. for 16 hours. Once the solution had cooled to room temperature, it was precipitated with ethyl ether (30 mL×3). As a result of drying the leftover residue under vacuum, 0.215 g of red solid in 77% yield was obtained. ¹H-NMR (400 MHz, D₂SO₄): 9.82-9.74 (m, 8H), 7.56 (NH), 5.58 (m, 4H), 4.94-4.45 (m, 20H), 3.82 (s, 24H). Exact mass calculated for C₄₄H₆₂N₈O₄: 760.97. Found: 761.43 (MH⁺ ion peak).

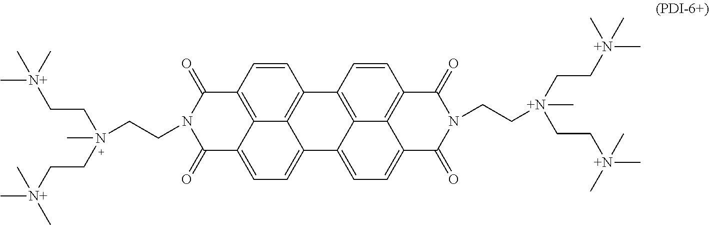

N¹,N^{1′}-((1,3,8,10-tetraoxo-1,3,8,10-tetrahydroanthra[2,1,9-def:6,5,10-d′e′f′]diisoquinoline-2,9-diyl)bis(ethane-2,1-diyl))bis(N¹,N²,N²,N²-tetramethyl-N¹-(2-(trimethylammonio)ethyl)ethane-1,2-diaminium) (PDI-6+): A mixture of 0.2 g of N¹,N^{1′}-((1,3,8,10-tetraoxo-1,3,8,10-tetrahydroanthra[2,1,9-def:6,5,10-d′e′f′]diisoquinoline-2,9-diyl)bis(ethane-2,1-diyl))bis(N1-(2-(dimethylammonio)ethyl)-N²,N²-dimethylethane-1,2-diaminium) and 10 mL of MeOH, and 130 mg Na₂CO₃ was stirred at room temperature for 12 h and then added methyl iodide (0.7 mL), heated at 60° C. for 12 h. The mixture was then cooled to room temperature, and then precipitated with ethyl ether (40 mL×3). The obtained solid was dried under vacuum to give a dark red solid PDI-6+ in 83% yield. ¹H NMR (400 MHz, D₂SO₄) δ 9.78 (d, J=24 Hz, 8H), 5.58 (s, 4H), 4.76-4.58 (m, 20H), 3.94 (s, 36H). Exact mass calculated for C₅₀H₇₄N₈O₄I₆: 1612.6. Found: 873.2 (M−6I⁻+Na⁺).

### Fluorescence Spectroscopic Studies of PDI-6+

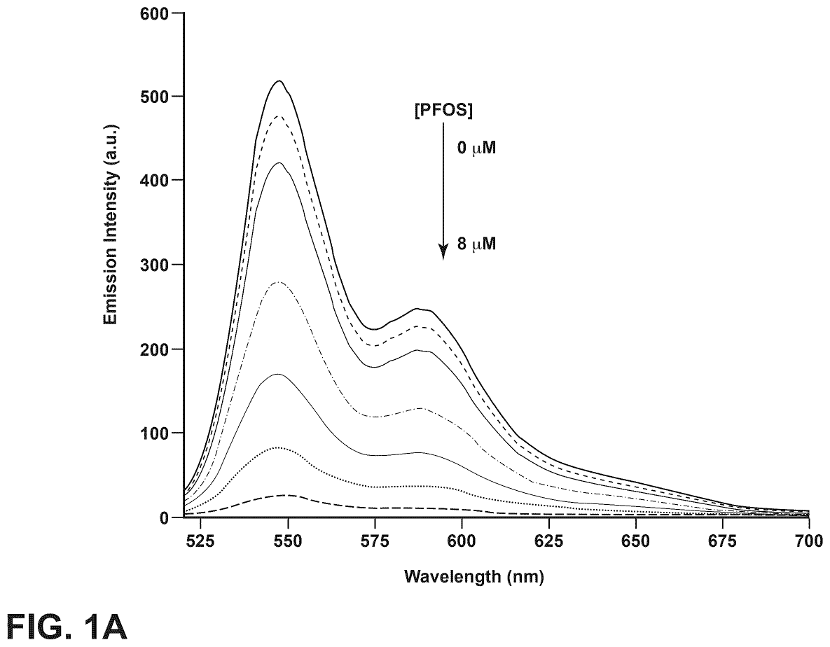

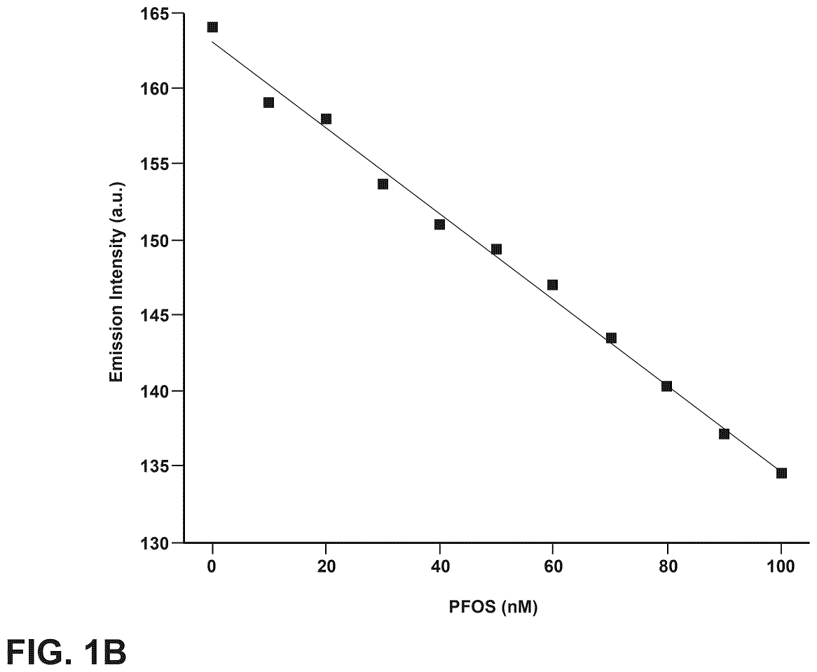

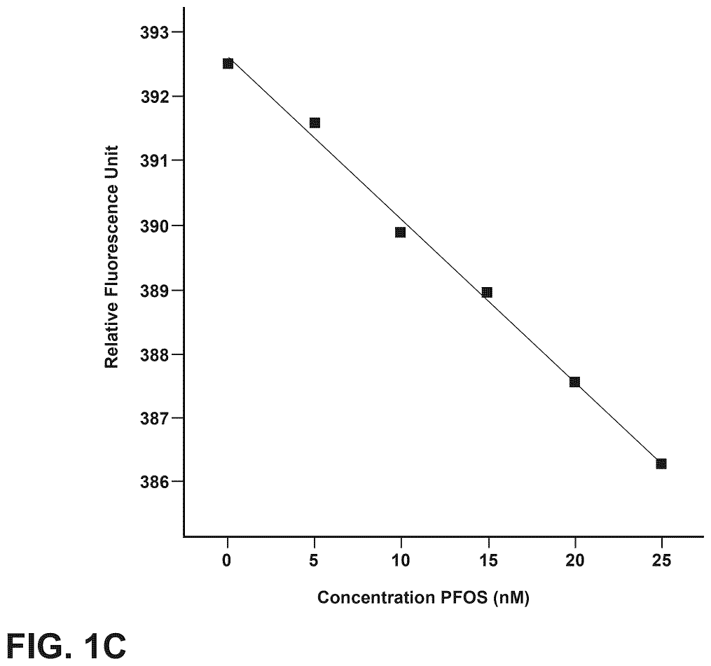

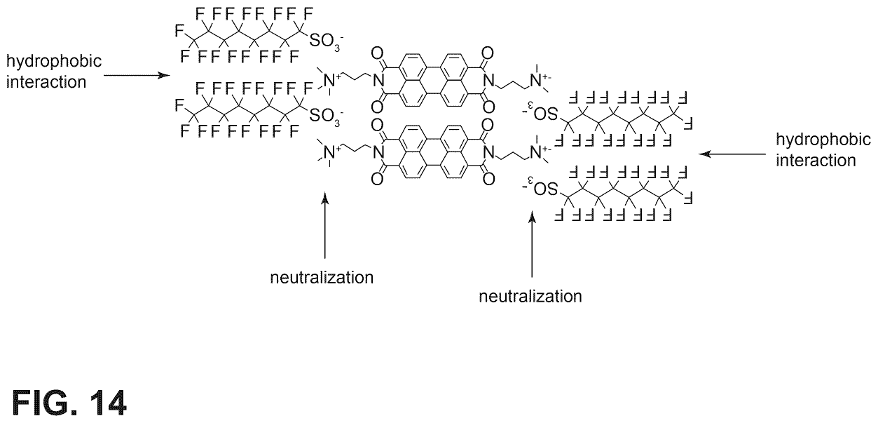

FIG. 1A illustrates a series of fluorescence emission spectra of an example PDI-6+ in the presence of varying concentrations of PFOS. In this example, a 2 μM PDI-6+ solution in water (synthesized according to Example 1) was excited at an absorbance wavelength of 480 and a very high fluorescence peak was observed at 550 nm. With the addition of perfluorooctane sulfonic acid (PFOS) from 0 to 8 μM, the emission intensity of this compound gradually decreased and became saturated with 8 μM of PFOS in aqueous media, as the electrostatic interaction between PFOS and PDI-6+ resulted in saturation. The decrease in emission intensity, particularly between 540 nm and 600 nm, illustrates the ability of PFOS to quench the emissions of PDI-6+. In this way, in the absence of PFOS, the molecularly dispersed PDI-6+ demonstrates a strong green emission in water devoid of PFOS, which is a signature for the molecularly dispersed (non-aggregated) form of the fluorophore, while in the presence of PFOS it will associate with PDI-6+ through electrostatic interaction and other interactions such as hydrogen bonding, hydrophobic interaction, thus resulting in aggregation of the fluorophore, which in turn causes fluorescence quenching, a scheme of which is illustrated in FIG. 14.

FIGS. 1B and 1C illustrate linear regression plots of fluorescence intensity of PDI-6+ in the presence of varying the concentrations of PFOS from 0 to 100 nM. A linear plot was used to calculate the limit of detect (LOD) using 3 k/slope, where k represents the standard deviation from 10 blank measurements of PDI-6+. To obtain the LOD of PFOS using PDI-6+, a linear regression of the fluorescence intensity of PDI-6+ at its maximum emissions wavelength of 547 nm (when excited at an absorbance wavelength of 480 nm) in the presence of varying amounts of PFOS (0-100 nM) was calculated. Based on fluorescence titrations using an Agilent Fluorescence Spectrophotometer, the detection limit for PDI-6+ for PFOS was as low as 14.7 nM (7.91 ppb) as shown in FIG. 1B.

FIG. 1C illustrates that it is possible to perform this analysis to measure the LOD using a portable fluorimeter. For these experiments, a linear regression plot was created of the fluorescence intensity of 100 nM of PDI-6+ at an emission wavelength of 540 nm (when excited at an absorbance wavelength of 485 nm) as a function of the concentration of PFOS (0-25 nM) as measured using an AquaFluor handheld fluorimeter. From the linear fitting, a limit of detection (LOD) of 22.0 nM (11.9 ppb) was calculated for PFOS using the portable fluorimeter. As compared with previous reports, the obtained LOD was lower (Table 1) than previously reported fluorophores. The fluorescence quantum yield (φ_{fl}) of PDI-6+(φ_{fl}=21%) and the PDI-6+PFOS (φ_{fl}=1.6%) complex was measured in comparison to Rhodamine 6G- a reference dye (φ_{fl}=0.95 in 0.1 μM in D.I. water). Accordingly, a linear calibration curve can be used to quantify an unknown concentration of PFOS in real water samples such as tap and drinking water using methods of the present disclosure. Specifically, fluorescence measurements of the disclosed solutions, compared to samples without PFOS, yield quenching efficiencies, which can be used to calculate a concentration by referencing the calibration curve.

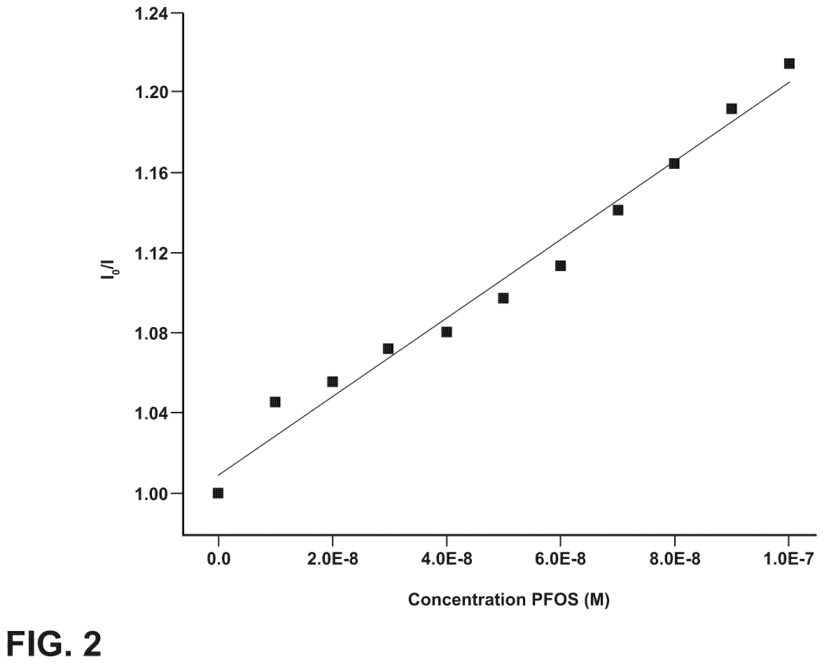

FIG. 2 illustrates interaction dynamics between PDI-6+ and the quencher in a Stern-Volmer plot. The Stern-Volmer constant, K_{SV}, value was calculated to be 1.96×10⁶ M⁻¹ for PFOS.

TABLE 1
Comparison of PDI-6+ with previous reports for PFOS detection

| | Analytical | | | |
|---|---|---|---|---|
| Sensor Probe | Technique | Solvent | Linear range | LOD |
| PDI-Pyr | Fluorescence | HEPES Buffer | 0.1-1.5 μM | 28 nM |
| Tetraphenylethylene- | Fluorescence | H₂O/THF | 0-3.0 μM | 47.3 nM |
| derived dual macrocycle | | | | |
| BowtieCyclophane | | | | |
| An erythrosin B-based | Fluorescence | BR buffer | 0-10 μM | 11.8 nM |
| probe | | | | |
| Guanidinocalix[5]arene- | Fluorescence | HEPES Buffer | 0-0.8 μM | 21.4 nM |
| based probe | | | | |
| A chitosan-mediated | Fluorescence | Pure water | 0-2 μM | 1.0 nM |
| An eosin Y-based probe | Fluorescence | BR buffer | 0-2 μM | 15 nM |
| Toluidineblue-based probe | Fluorescence | CA—NaCA buffer | 0-20 μM | 4.2 nM |
| | | solution | | |
| PDI-6+ | Fluorescence | Pure water | 0-100 nM | 14.7 nM |

### Absorption Studies of PDI-6+

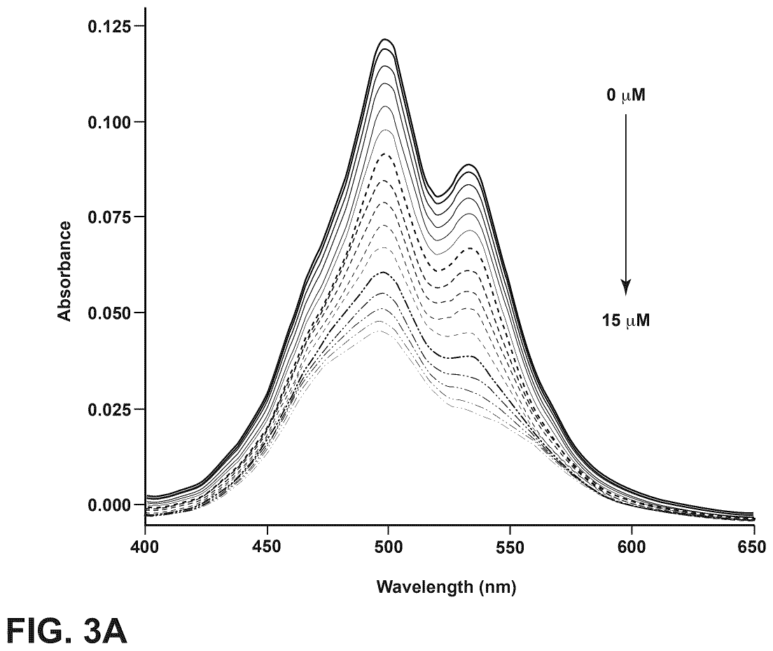

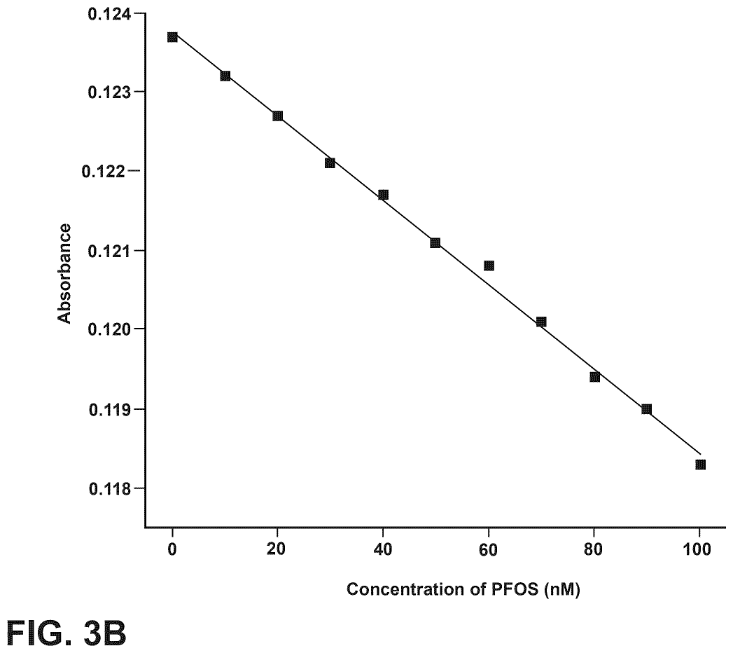

FIG. 3A illustrates a UV-vis absorption spectrum of PDI-6+, which showed peaks at 480 nm and 540 nm. The addition of PFOS (from 0-15 μM) to a 4 μM PDI-6+ solution gradually decreased the absorption peak. FIG. 3B illustrates a linear calibration plot of the absorbance at 499 nm versus concentration of PFOS (0-15 μM). The LOD was determined to be 8.46 nM (4.55 ppb), which is low compared with previous findings. Additionally, the color of the [PDI-6+PFOS] solution turned almost colorless, which could be seen with the naked eye, indicating the formation of PFOS adducts with PDI-6+.

### Selectivity and Interference Studies of PDI-6+

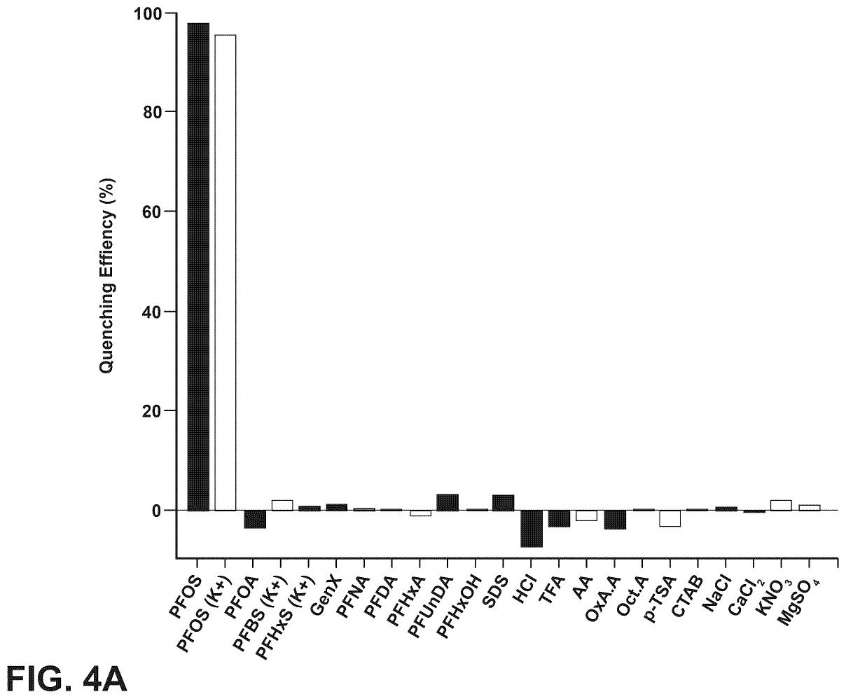

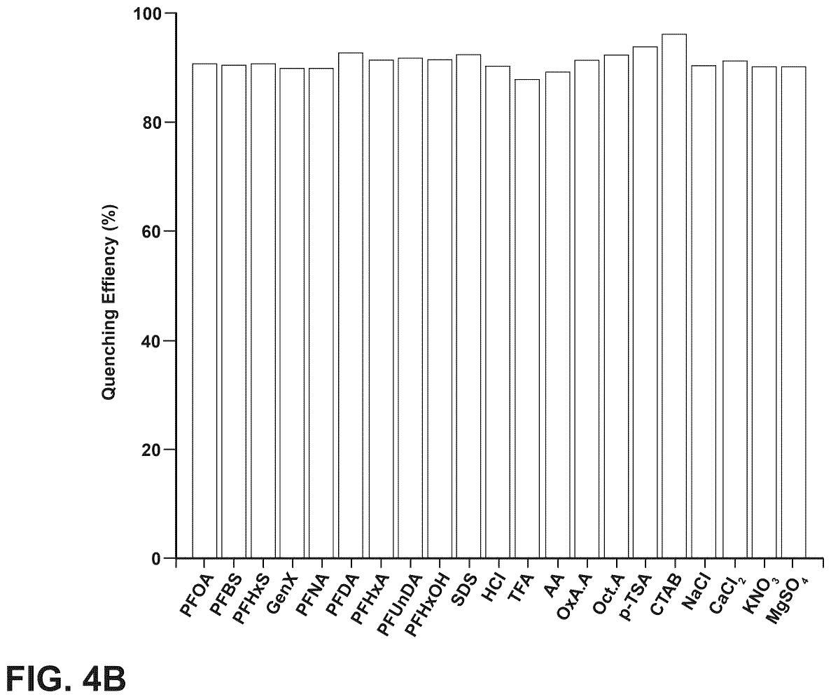

FIG. 4A illustrates the quenching efficiency (e.g., a decrease in intensity relative to the original intensity) of 2 μM PDI-6+ with a plurality of different analytes, each at a concentration of 10 μM. The fluorescence quenching efficiency of the PDI-6+ was calculated in the experiment as the percent decrease in intensity of the PDI-6+ as measured at an emission wavelength of 547 nm (when excited at a wavelength of 480 nm) in the presence of the various other analytes relative to the intensity of the fluorescence intensity of the PDI-6+ in the absence of the corresponding analyte. The analytes tested included: potassium perfluorooctane sulfonate (PFOS), perfluorobutane sulfonic acid (PFBS), perfluorohexanesulponic acid (PFHxS) GenX, perfluorohexanoic acid (PFHxA), perfluorohexanol (PFOHOH), perfluorooctanoic acid (PFOA) perfluoronononanoic acid (PFNA), perfluorodecanoic acid (PFDA), perfluoroundecanoic acid (PFUDA), sodium dodecyl sulfate (SDS), acetic acid (AA), trifuoroacetic acid (TFA), oxalic acid (OA), lauric acid (LA), calcium chloride (CaCl₂) magnesium chloride (MgCL₂), sodium chloride (NaCl), potassium chloride (KCl), ethylenediaminetetraacetic acid (EDTA), 4-nitro benzene (NB) and phenol. There was little to no distinct change in the emission spectrum of PDI-6+(2 μM in DI water) after 10 μM of other analytes were added, thereby illustrating that the selectivity of PDI-6+ with PFOS is not affected by the presence of the tested analytes.

Now referring to FIG. 4B, further interference studies were performed to investigate the changes in the emission spectrum for PDI-6+ upon detection of PFOS. The interference studies showed the ability of a 10 μM solution of PFOS to quench the fluorescence emissions of 2 μM solution of PDI-6+ at 547 nm, when excited at 480 nm, in water with the presence of various other analytes, each at a 30 μM solution. When PFOS was added to the sensor PDI-6+ solution, the emission intensity at 547 nm of the mixture decreased gradually. In the presence of other analytes, there was no apparent change in color or fluorescence intensity, thus again showing the selectivity of PDI-6+ towards PFOS.

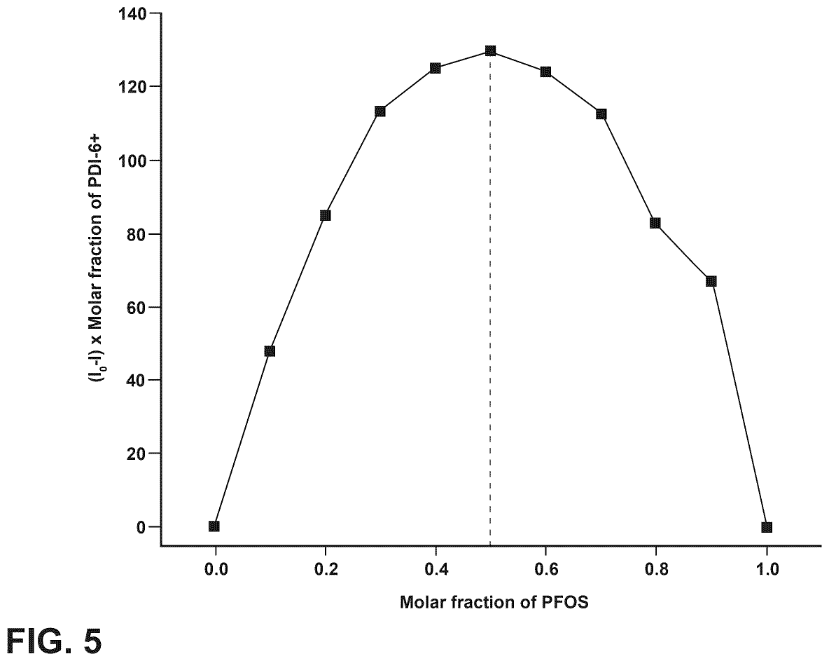

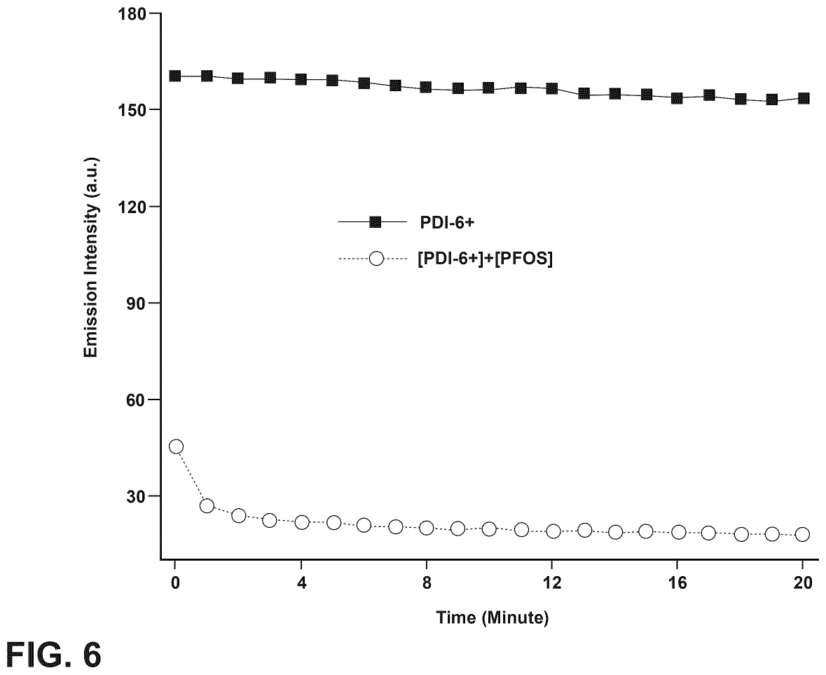

Turning now to the Job's plot analysis shown in FIG. 5, the stoichiometric interaction between PDI-6+ and PFOS is illustrated. As shown in FIG. 6, the curve having a maximum at 0.5 mole fraction suggests that sensor PDI-6+ and PFOS may form a complex of a 1:1 ratio. As a result of PFOS electrostatic interaction with cationic PDI-6+, [PDI-6]+[PFOS] complex was formed. The combined concentration of PDI-6+ and PFOS was maintained at 1 μM for the Job's plot measurement.

### Effect of Response Time of PDI-6+

For the detection of PFOS by sensor PDI-6+, the impact of response time on the fluorescence intensities of PFOS was examined. According to FIG. 6, the fluorescence emission intensity of 100 nM PDI-6+ at an emission wave length of 547 nm (when excited at an absorbance wavelength of 480 nm) in the presence of 10 μM PFOS, decreased immediately due to the [PDI-6+]+[PFOS] complex, and was completely quenched within 3 minutes, and then stabilized. This is compared to the fluorescence emission intensity of 100 nM PDI-6+ at an emission wavelength of 547 nm/(when excited an absorbance wavelength of 480 nm) in the absence of 10 μM PFOS, showing no decrease with respect to time. In the results, it was found that the PDI-6+ could be used to detect PFOS in real time.

### Effect of Temperature on PDI-6+

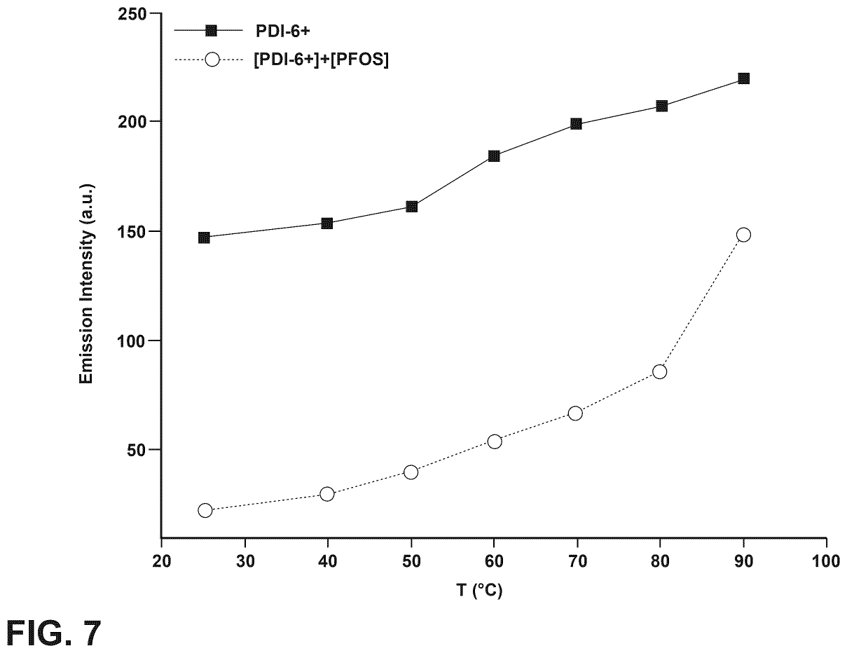

The effects of reaction temperatures ranging from 25° C. to 90° C. on the fluorescence of the experimental system. For the purposes of this experiment, the fluorescence emission intensity was studied of 100 nM PDI-6+ at an emission wavelength of 547 nm (when excited at an absorbance wavelength of 480 nm) in the absence of PFOS and in the presence of 10 μM PFOS as a function of temperature. As shown in FIG. 7, PDI-6+ and its [PDI-6+]+[PFOS] systems have almost no effect when the temperature is below 40° C. As illustrated in FIG. 7, when the temperature exceeds 40° C., the fluorescence intensity sharply increases, likely due to PDI-6+ increased solubility. As the temperature increases, molecules move more quickly, likely leading to the dissociation of the [PDI-6+]+[PFOS] complex, followed by the recovery of fluorescence in PDI-6+. As a result of this phenomenon, the [PDI-6+]+[PFOS] complex is capable of sensing temperature. In the end, 25° C. is selected as the reaction temperature.

### Effect of pH of PDI-6+

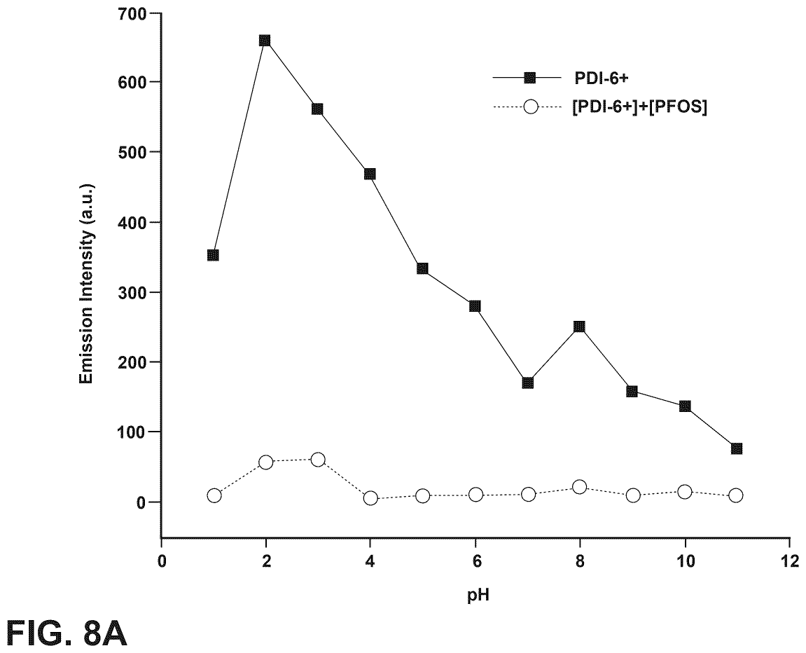

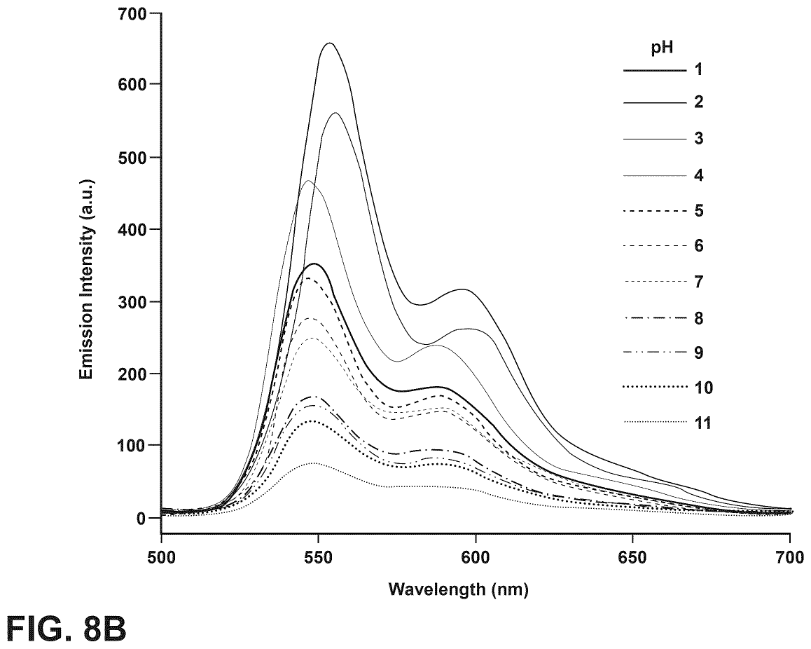

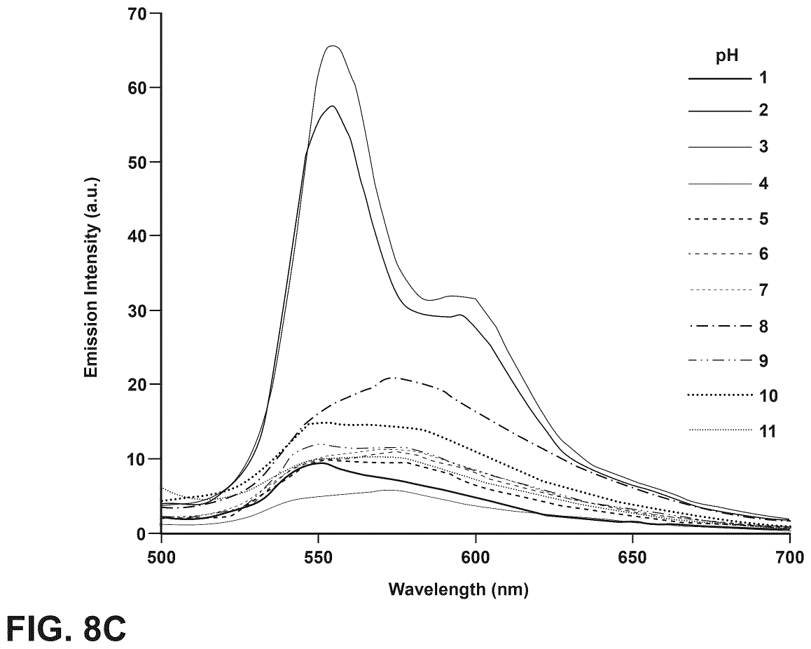

A wide pH range of 1-11 was tested for the pH-dependence of PDI-6+ alone and the [PDI-6+]+{PFOS] complex. FIG. 8A shows a plot of the fluorescence emission intensity of 1 μM PDI-6+ at an emission wavelength of 547 nm (when excited at a wavelength of 480 nm) in the absence of PFOS and in the presence of 10 μM PFOS as a function of pH. FIG. 8B shows a series of fluorescence emission spectra of 1 μM PDI-6+ at an emission wavelength of 547 nm (when excited at an absorbance wavelength of 480 nm) at varying pH levels. FIG. 8C shows a series of fluorescence emission spectra of 1 μM PDI-6+ at an emission wavelength of 547 nm (when excited at an absorbance wavelength of 480 nm) in the presence of 10 μM PFOS at varying pH levels. As shown, in a pH range of 2 to 11, PDI-6+ fluorescence exhibited strong emission, but emission intensity decreased significantly. Further, as shown, the fluorescence intensity solution of the [PDI-6+]+[PFOS] complex changed slightly from pH 1.0 to 3.0 in the presence of 10 μM PFOS, but not from pH 4.0 to 11.

### Effect of Ionic Strength of PDI-6+

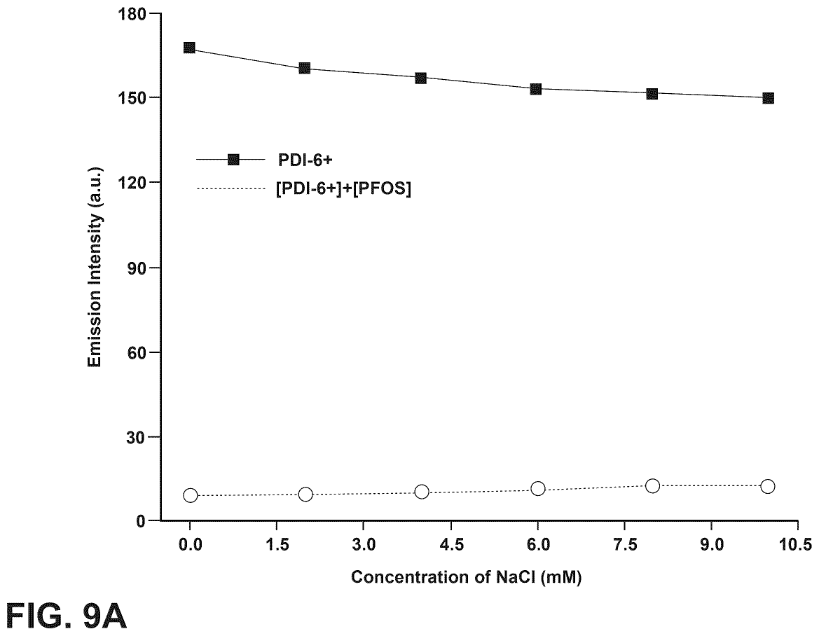

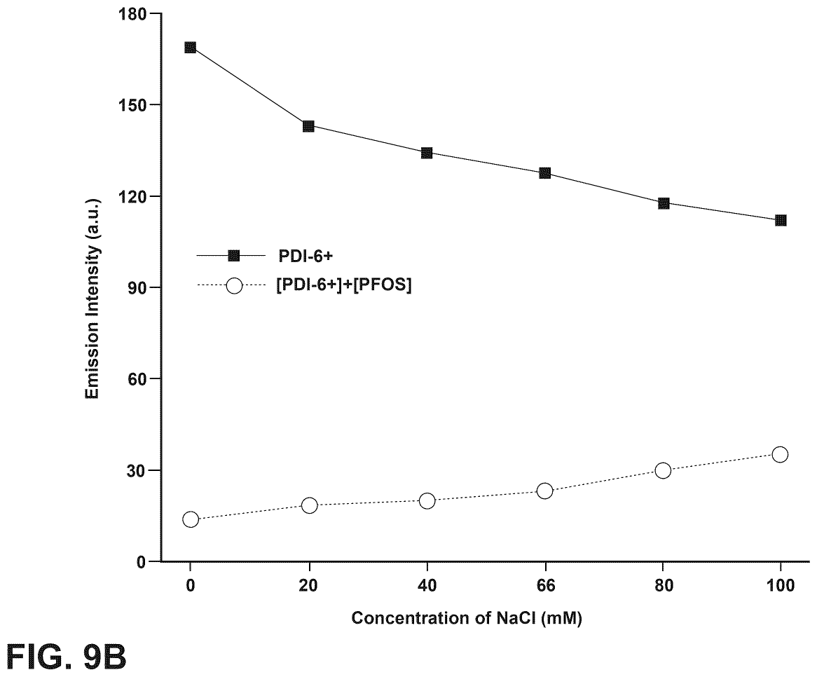

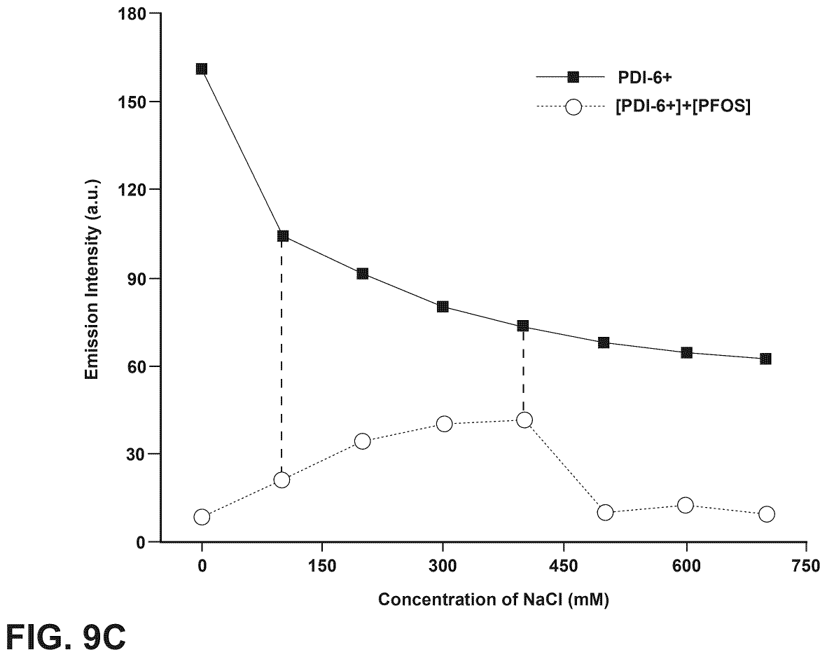

The influence of salt was also examined with various concentrations of sodium chloride (0-10 mM) while keeping PDI-6+ and the [PDI-6+]+[PFOS] complex at fixed concentrations. FIG. 9A shows the fluorescence emission intensity of 100 nM PDI-6+ at an emission wavelength of 547 nm (when excited at an absorbance wavelength of 480 nm) in the absence of PFOS and also in the presence of 10 μM PFOS as a function of ionic strength (i.e., 0-10 mM NaCl). As shown, in the presence of 10 mM salt, the fluorescence intensity of PDI-6+ showed a small change (11%). Thus, both PDI-6+ and the [PDI-6]+[PFOS] complex appeared to be stable in conditions maintained by the complex, and therefore, no ionic strength adjustment was required.

FIG. 9B shows the fluorescence emission intensity of 100 nM PDI-6+ at an emission wavelength of 547 nm (when excited at a wavelength of 480 nm) in the absence of PFOS and also in the presence of 10 μM PFOS as a function of ionic strength (i.e., 0-100 mM NaCl). As shown, at higher salt concentrations (0-100 mM NaCl), approximately 34% of quenching efficiency was induced for PDI-6+. In the case of the [PDI-6+]+[PFOS] complex, a significant increase in emission intensity is shown in FIG. 9B and also in the region to the left of the first vertical line in FIG. 9C. This result may indicate that the sodium and chloride ions interfere with PFOS and electrostatic interaction between the sulfonic group of PFOS and PDI-6+ was weakened, resulting in enhancement of the fluorescence (FL) intensity. Still referring to FIG. 9C, the fluorescence emission intensity of 100 nM PDI-6+ at an emission wavelength of 547 nm (when excited at a wavelength of 480 nm) in the absence of PFOS and in the presence of 10 μM PFOS as a function of ionic strength (i.e., 0-700 mM NaCl) was plotted. As shown, up to 400 mM NaCl, the fluorescence intensity of PDI-6+ significantly decreased. On the other hand, the [PDI-6+]+[PFOS] complex follows an increasing trend. Further, as shown, over 400 mM NaCl, there is no change in fluorescence intensity, which can be seen in the region to the right of the second vertical line in FIG. 9C.

### Paper Strip Application of PDI-6+

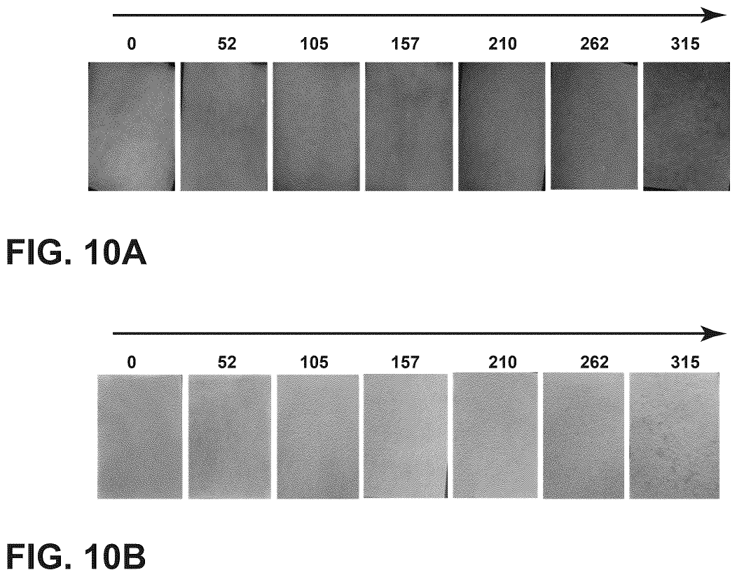

For the evaluation of the practical applicability of PDI-6+ as a sensor, Whatman® filter paper strips were immersed in aqueous PDI-6+ solution (0.11 mM) and then dried for 15 minutes in a hot-air-oven and then exposed to either UV light at 365 nm (FIG. 10A) or normal light (FIG. 10B). Different concentrations of PFOS were prepared in distilled (D.I.) water. As shown in FIG. 10A, the coated paper strips, when immersed in 0 to 315 μM PFOS solutions, immediately changed from yellow to colorless (e.g., yellow coloring is absent) when exposed to the UV light. As a result, PDI-6+ on paper strips could be useful for detecting PFOS in environmental water with high selectivity and in real-time with the use of a UV light.

Detection of PFOS using RGB Method with PDI-6+

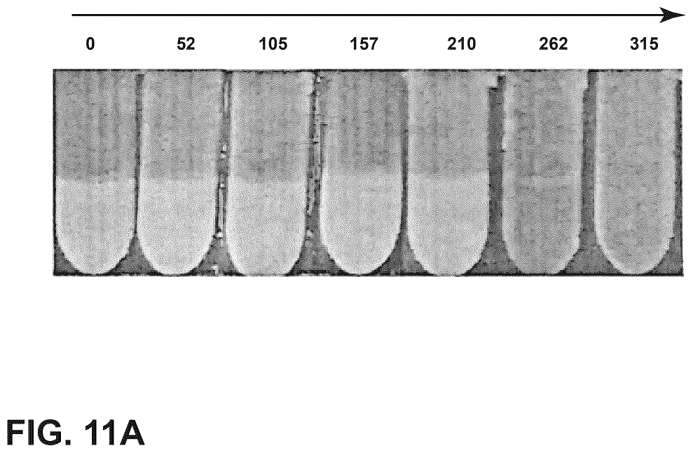

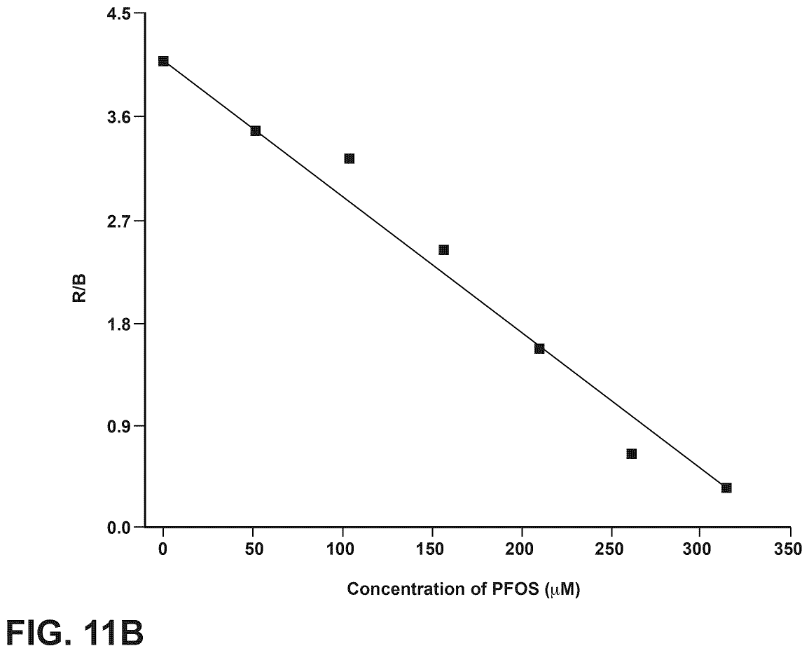

By recording an RGB value of samples of the color change can be monitored. Using a back camera of a Redmi Note 5, the RGB (red, green, blue) values of vials containing PDI-6+ solutions (FIG. 11A) were measured in the absence and presence of different concentrations of PFOS using a mobile APP (Color Picker). As a result, the colorimetry analysis by smartphone provided a simple solution for quantifying PFOS when naked-eye observation was insufficient. As shown in FIG. 11B, RGB values of the vials of FIG. 11A that contained PDI-6+ (0.11 mM) solution with varying concentrations of PFOS (0 to 315 μM) were plotted against the concentrations of PFOS. The linearity range was R²=0.9952 and the estimated LOD was 13.1 μM (7.10 ppm).

Analysis of PFOS with PDI-6+ in Environment Water Samples

To demonstrate the practical utility of PDI-6+ for detection of PFOS, different environmental water samples were tested, including drinking water, tap water, Parleys Creek water, and pond water. Water samples were collected from the University of Utah and Sugar House Park (Salt Lake City, Utah, U.S.A). Each sample was spiked with 100 nM of PFOS, and the experiment was repeated three times. As shown in Table 2, PDI-6+ was able to detect the spiked PFOS with good recovery, which ranged from 93.2% to 110.4% and had a low RSD (0.33% to 0.47%) for determination of the PFOS in the real water samples. For detecting PFOS in drinking water, tap water, Parley's Creek water, and pond water, probe PDI-6+ showed good agreement with that in D.I. water, which showed the formation of the PDI-6+/PFOS complex. As a result, PDI-6+ is an excellent probe for sensing PFOS in environmental water samples and confirms that it has great potential.

TABLE 2
Detection of PFOS with PDI-6+ in environmental water samples

| Environmental | Spiked | PFOS Found | | | R.S.D. (%) |
|---|---|---|---|---|---|
| Water Sample | PFOS (nM) | (nM) | Recovery (%) | Error (%) | (n = 3) |
| Drinking Water | 100 | 94.3 ± 3.81 | 94.3 | −5.74 | 0.450 |
| Tap Water | 100 | 95.3 ± 3.37 | 95.3 | −4.70 | 0.330 |
| Pond Water | 100 | 110.4 ± 4.47 | 110.4 | 10.4 | 0.370 |
| Parleys Creek | 100 | 93.2 ± 4.03 | 93.2 | −6.83 | 0.460 |
| Water | | | | | |

### Example 2: Determination of Polyfluorooctane Sulfonic Acid (PFOS) with Fluorescent Probe PDI-2+

### Abbreviations

PTCDA is perylene tetracarboxylic dianhydride and is used as starting material for synthesizing PDI-2+ and PDI-6+, which contain two and six quaternized amines, respectively;
DMF is dimethylformamide;
NMR is nuclear magnetic resonance;
TLC is thin-layer chromatography;
eq. or equiv. is equivalents;

### Exemplary Synthesis of PDI Intermediates

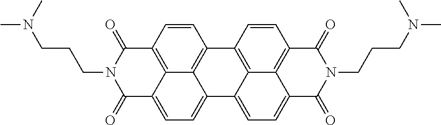

2,9-bis(3-(dimethylamino)propyl)anthra[2,1,9-def:6,5,10-d′e′f′]diisoquinoline-1,3,8,10(2H,9H)-tetraone: A flask was filled with 0.2 g of perylene tetracarboxylic dianhydride (PTCDA) (0.51 mmol). After filling the flask with nitrogen several times, 3-dimethylaminopropylamine (0.19 mL, 1.5 mmol) and 5 mL of DMF were injected. The mixture was then heated overnight at 130° C. After filtering, the crude product was washed with deionized (D.I.) water and ethanol. The yield of red solids obtained after drying in vacuum was 78%.¹H-NMR (400 MHz, CF₃ COOD): 7.77 (ArCH, 4H), 7.48 (ArCH, 4H), 4.12 (CH₂, 4H), 3.38 (CH₂, 4H), 3.04 (CH₃, 4H), 2.28 (CH₂, 2H).

### Exemplary Synthesis of PDI-Based Fluorophores

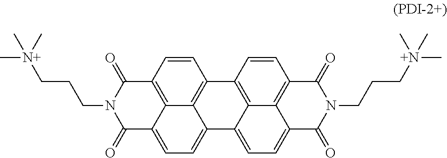

3,3′-(1,3,8,10-tetraoxo-1,3,8,10-tetrahydroanthra[2,1,9-def:6,5,10-d′e′f′]diisoquinoline-2,9-diyl)bis(N,N,N-trimethylpropan-1-aminium) (PDI-2+): In 10 mL of dry toluene, 2,9-bis(3-(dimethylamino)propyl)anthra[2,1,9-def:6,5,10-d′e′f′]diisoquinoline-1,3,8,10(2H,9H)-tetraone (0.1 g, 0.18 mmol) and iodomethane (0.44 mL, 7.2 mmol) were dissolved. Following 4 hours of refluxing in N₂ atmosphere at 60° C., the solid was filtered and washed with ether. Pure product yielded a brown solid (83%). ¹H-NMR (400 MHz, DMF-d₇): 9.03 (ArCH, 4H), 8.70 (ArCH, 4H), 4.48 (CH₂, 2H), 3.97 (CH₂, 4H), 3.54 (CH₃, 18H), 2.48 (CH₂, 2H).

The synthesis of PDI-CI-2+ was carried out using the same method as for PDI-2+, but with 1,6,7,12-tetrachloro-substituted PTCDA as the precursor instead.

### Fluorescence Spectroscopic Studies of PDI-2+

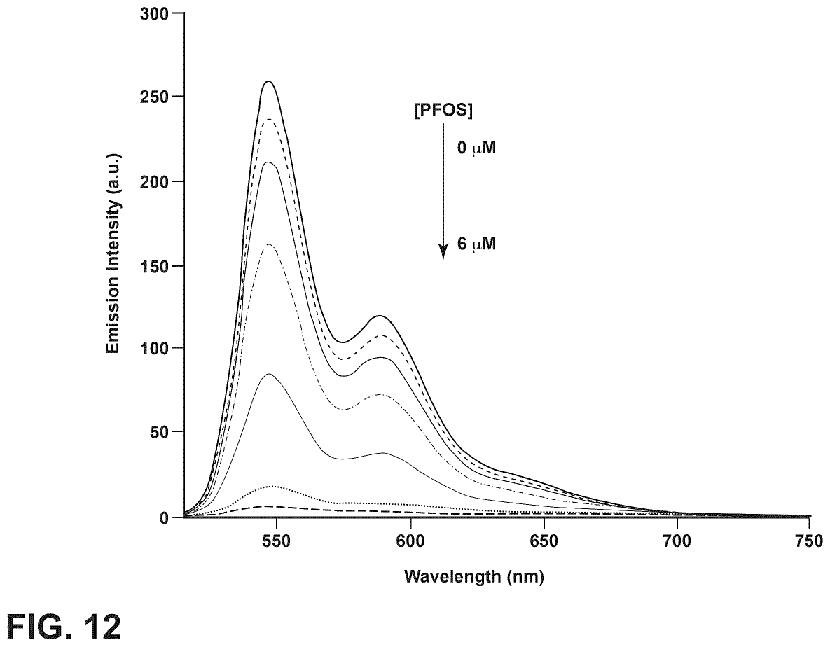

FIG. 12 shows fluorescence spectra of PDI-2+ (2 μM) when excited at an absorbance wavelength of 500 nm in the presence of PFOS having different concentrations in pure aqueous medium to determine PFOS sensing by PDI-2+. With increasing concentration of PFOS from 0 to 6 μM, the emission intensity of PDI-2+ drastically quenched up to 98%. This result clearly demonstrates that PDI-2+ has a large tendency to interact with PFOS.

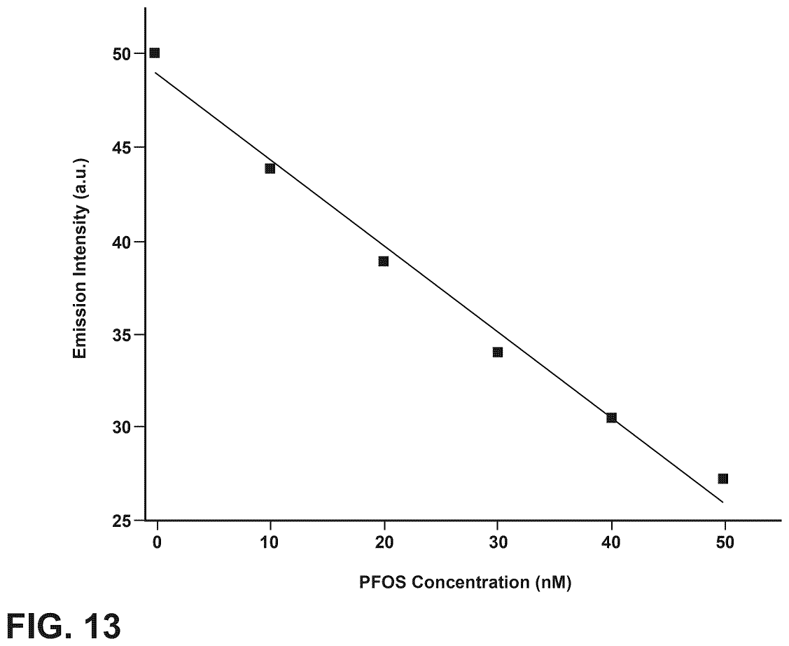

FIG. 13 shows a linear regression plot of the fluorescence intensity of 10 nM PDI-2+ in water at an emission water at an emission wavelength of 547 nm (when excited at an absorbance wavelength of 500 nm) as a function of the concentration of PFOS (0-50 nM). The fluorescence titration experiments were carried out with PDI-2+ by adding PFOS solution in the concentration range of 0-100 nM to determine the limit of detection (LOD). A linear fluorescence response was observed in this lower concentration range of PFOS. This linear response could be used to quantify the unknown concentration of PFOS in real samples. From the linear fitting, the LOD was found to be 7 nM (3.7 ppb) by using the IUPAC 36 criterion.

Without wishing to be bound by theory, FIG. 14 shows a schematic illustration of PFOS detection mechanism by PDI-2+ molecules. The cationic head of PDI-2+ can interact with the sulfonate group of PFOS. Further driving the complexation of PDI-2+ with PFOS are the hydrophobic interactions between the fluorinated chains of the PFOS molecules. All such interactions facilitate the fluorescence quenching (e.g., aggregation) caused by π-π stacking between the PDI core of PDI-2+ molecules. Accordingly, the aggregation is a result of several synergistic intermolecular interactions between PFOS and the PDI-based fluorophore, including electrostatic attraction between the head groups, π-π stacking between PDI backbones, and hydrophobic association between the perfluoroalkyl chains of PFOS. In the aggregate state, the π-π stacking quenches the emission of PDI, which is a phenomenon of aggregation induced quenching for n-conjugated fluorophores. It is believed that a competitive balance between the electrostatic interactions and π-π stacking plays a crucial role in determining optimal sensing performance.

### Selectivity and Interference Studies of PDI-2+

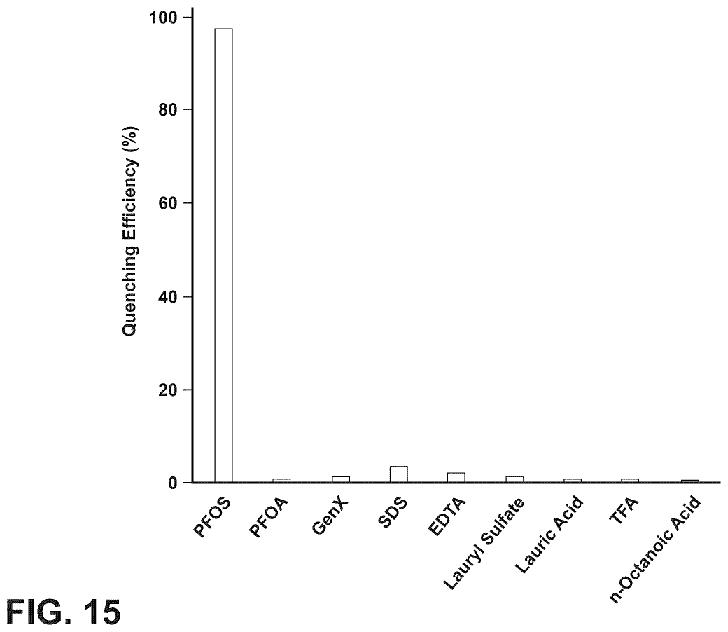

To assess the selectivity of PDI-2+ probe toward PFOS, commonly used other PFAS molecules (PFOA, GenX), surfactant (SDS), acids (trifluoroacetic acid, lauric acid, octanoic acid), relevant molecules (EDTA, lauryl sulfate), metal salts (NaCl, KCl, CaCl₂, MgCl₂) were treated with a PDI-2+ probe. Each analyte was introduced at a concentration of 6 μM to a 2 μM PDI-2+ water solution. The fluorescence quenching efficiency calculated in the experiment was the percent decrease in intensity of the PDI-2+ as measured at an emission wavelength of 547 nm (when excited at an absorbance wavelength of 500 nm) in the presence of the various analytes relative to the intensity of the fluorescence intensity of the PDI-2+ in the absence of the analyte. As shown in FIG. 15, except for PFOS, other analytes show an insignificant effect on fluorescence quenching of PDI-2+ probe, which suggests that PDI-2+ is highly selective towards PFOS in a pure aqueous medium.

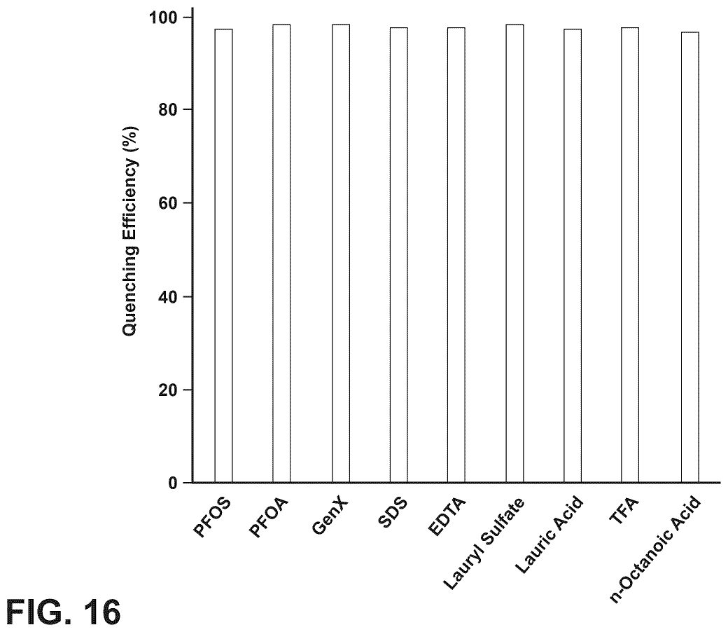

To determine the effect of a coexistence of other interfering species on the sensing ability of PDI-2+, competitive experiments were carried out for PFOS sensing in the presence of the interfering molecules or ions. FIG. 16 illustrates that the presence of interfering species has minimal impact on the sensing ability of PDI-2+ in detecting PFOS (where the first bar in the chart labeled “PFOS” shows the quenching efficiency of PFOS when in the absence of any other interfering species, and the remaining bars labeled with other species shows the effect of those species on the quenching efficiency of PFOS). The interference testing compares the ability of 6 mM PFOS to quench 2 μM PDI-2+ in water in the presence of the various other analytes, each at a concentration of 6 μM. These findings strongly indicate the remarkable selectivity of the PDI-2+ probe for PFOS detection.

### Absorption Studies of PDI-2+

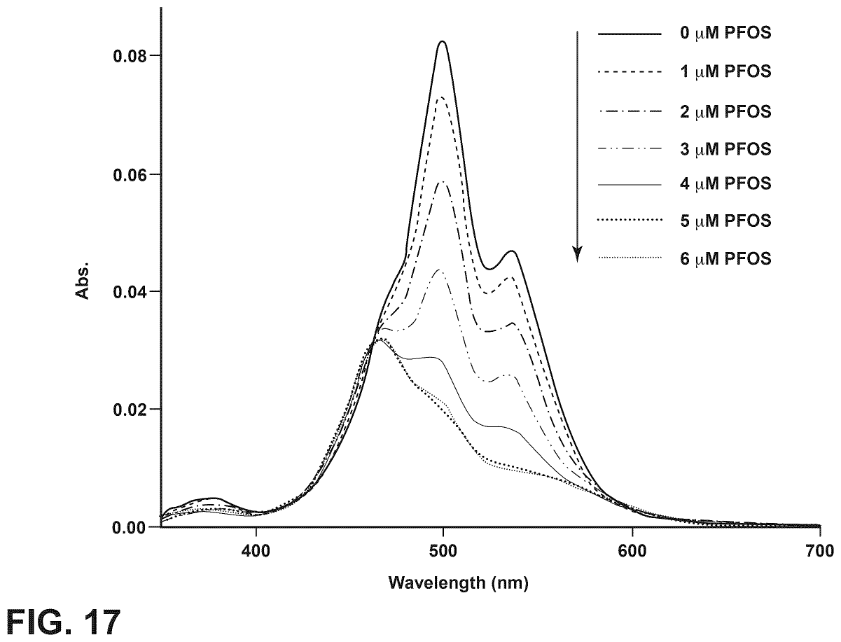

UV-Vis spectroscopy studies were used for the PDI-based sensor molecules to understand the aggregation behavior. As shown in FIG. 17, the absorption maxima at 500 nm, corresponding to 0→1 transition rapidly reduced, whereas the absorption peak corresponding to 0→0 transition at 536 nm disappeared in presence of 3 equivalents (6 μM) of PFOS. A new isosbestic point was observed at 463 nm. These results suggest formation of a rapid nano aggregate via electrostatic interaction between PFOS and PDI-2+ probe molecules.

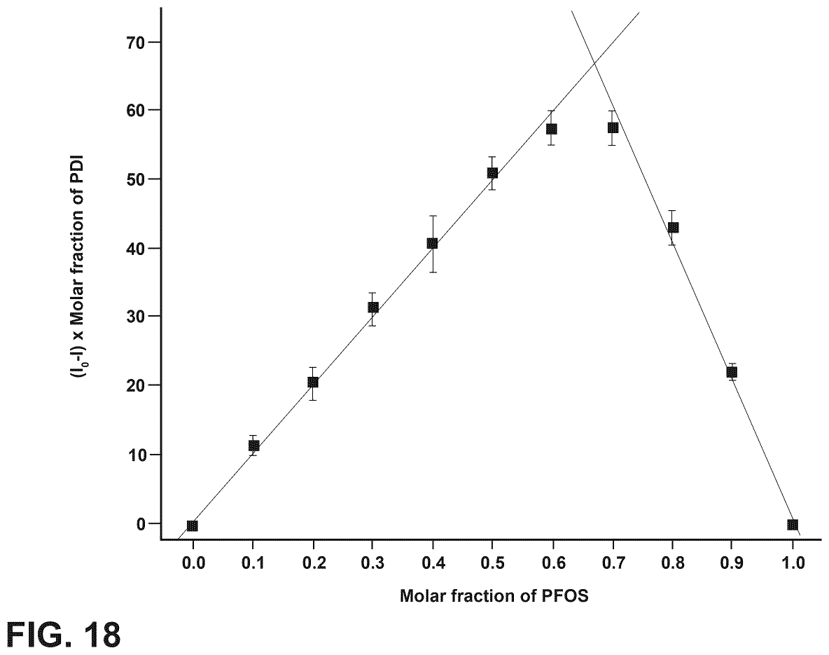

FIG. 18 shows a Job plot showing the stoichiometry of the complex between PDI-2+ and PFOS. The combined concentration of PDI-2+ and PFOS was maintained at 2 mM for the Job's plot measurement. The Job's plot confirms the formation of 2:1 complexation between PFOS and PDI-2+ molecules.

### Example 3: Covalent Attachment of PDI Derivatives to Silica

### Abbreviations

KOH is potassium hydroxide;
APTMS is (3-Aminopropyl)trimethoxysilane;
NMR is nuclear magnetic resonance;
TLC is thin-layer chromatography;
eq. or equiv. is equivalents;
min or min. is minute(s);
h or hr. is hour(s);
rt, RT, or r.t. is room temperature; and
rpm is rotations per minute.

### Example Synthesis of Example Perylene Diimide

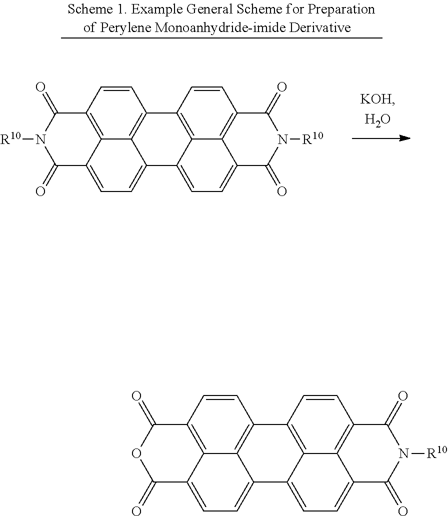

In the scheme above, —R¹⁰ generically includes -L¹-N(R¹)₃, where L¹, at each occurrence, is independently C₁₋₆alkylene or C₁₋₃alkylene-C═C—C₁₋₃alkylene; R¹, at each occurrence, is independently hydrogen, C₁₋₆alkyl, or C₁₋₆alkylene-N(R^{Y})ₙ, wherein n=1−3, and R^{Y}, at each occurrence, is independently hydrogen or C₁₋₄alkyl. The scheme above should not be construed as limiting in any way to the generalized description of the methods for covalently attaching the PDI-based fluorophores of the present disclosure to a solid support. In this example, the conjugated core is perylene, and the perylene monoanhydride-monoimide derivative was synthesized from perylene diimide via partial hydrolysis.

Exemplary perylene monoanhydride-monoimides were produced using the PDI-2+ and PDI-6+ that were both described above in Example 2 and Example 1, respectively. A solution of 35 mg of KOH (0.6 mmol) in 3 mL of water was prepared at 60° C. by dissolving the KOH in water. When the solution reached room temperature, 30 mg of the PDI-2+ or PDI-6+ were added (in separate reactions) under N₂, and then the mixture was stirred at 90° C. for 1.5 h. After cooling to room temperature, each solution turned brown. 3 mL of 2 M H₂SO₄ was added slowly to each solution and stirred at room temperature for 0.5 h. Following centrifugation (20 rpm, 5 minutes), a brown precipitate formed.

From the reaction that utilized PDI-6+ as a reactant, the following product was obtained:

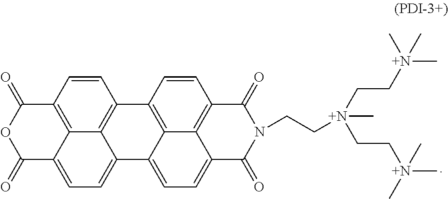 [SMILES: C[N+](C)(C)CC[N+](C)(CCN1C(=O)c2ccc3c4ccc5c6c(ccc(c7ccc(c2c37)C1=O)c64)C(=O)OC5=O)CC[N+](C)(C)C]

Thus, the final compound is a perylene monoanhydride-monoimide derivative.

N¹,N¹,N1,N2-tetramethyl-N2-(2-(1,3,8,10-tetraoxo-1,3,8,10-tetrahydro-9H-isochromeno[6′,5′,4′:10,5,6]anthra[2,1,9-def]isoquinolin-9-yl)ethyl)-N2-(2-(trimethylammonio)ethyl)ethane-1,2-diaminium (PDI-3+).¹H-NMR (400 MHz, CF₃COOD): 8.83 (ArCH, 8H), 4.86 (CH₂, 4H), 4.35 (CH₂, 8H), 3.41 (CH₃, 21H). Exact mass calculated for C₃₇H₄₁N₄O₅: 621.3060 Found: 622.3023 (MH⁺ ion).

From the reaction that utilized PDI-2+ as a reactant, the following product was obtained:

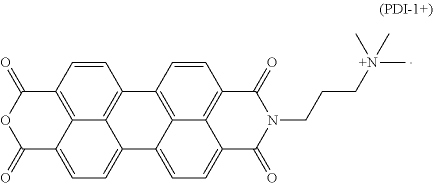 [SMILES: C[N+](C)(C)CCCN1C(=O)c2ccc3c4ccc5c6c(ccc(c7ccc(c2c37)C1=O)c64)C(=O)OC5=O]

N,N,N-trimethyl-3-(1,3,8,10-tetraoxo-1,3,8,10-tetrahydro-9H-isochromeno[6′,5′,4′:10,5,6]anthra[2,1,9-def]isoquinolin-9-yl)propan-1-aminium (PDI-1+). ¹H-NMR (400 MHz, CF₃COOD): 8.96-8.72 (ArCH, 8H), 4.58-4.52 (CH₂, 2H), 3.75-3.73 (CH₂, 2H), 3.29 (CH₃, 9H), 2.58-2.52 (CH₂, 2H). Exact mass calculated for C₃₀H₂₃N₂O₅: 491.1607. Found: 491.1580.

### Example Synthesis of Example Perylene Monoanhydrides Attached to Silica

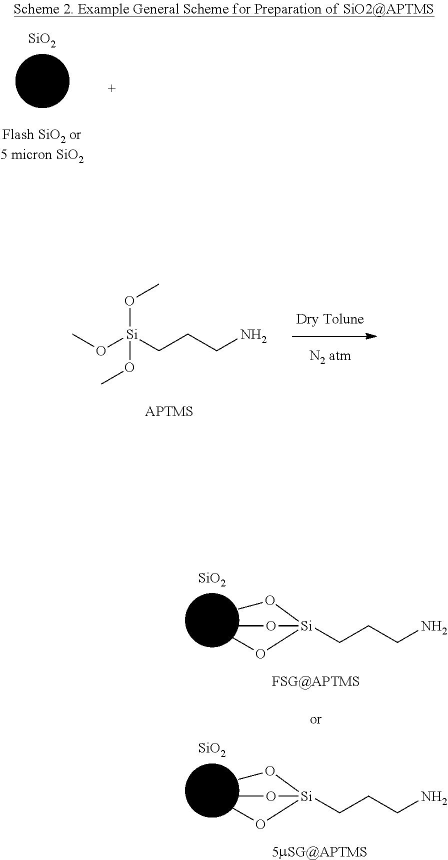

Two different types of SiO₂ were refluxed under N₂ with 3-aminopropyltrimethoxysilane (APTMS):

Flash SiO₂ (FSG) having a particle size of 60 μm and an average surface area of 500 m²/g; 5 μm spherical SiO₂ (5 μSG) having a particle size of 5 μm and an average surface area of 480 m²/g.

Both forms of SiO₂ were activated at 130° C. for 3 hours before any further reaction. 4.5 g (0.018 mol) of activated FSG was placed under N₂ and filled with 50 mL of dry toluene. To that 10.5 mL of APTMS (0.06 mol) was added and refluxed for 72 hrs. After cooling, the reaction mixture was washed with toluene (4×50 mL) followed by ethanol (15×4 mL). The resultant product (FSG@APTMS) was dried under vacuum oven at 60° C. overnight.

In a similar fashion, 1 g (0.004 mol) of activated 5 μSG was refluxed under N₂ with 2.4 mL (0.014 mol) of APTMS for 72 hrs. After cooling, the reaction mixture was washed with toluene (4×10 mL) followed by ethanol (4×10 mL). The resultant product 5 μSG@APTMS was dried under vacuum oven at 60° C. overnight.

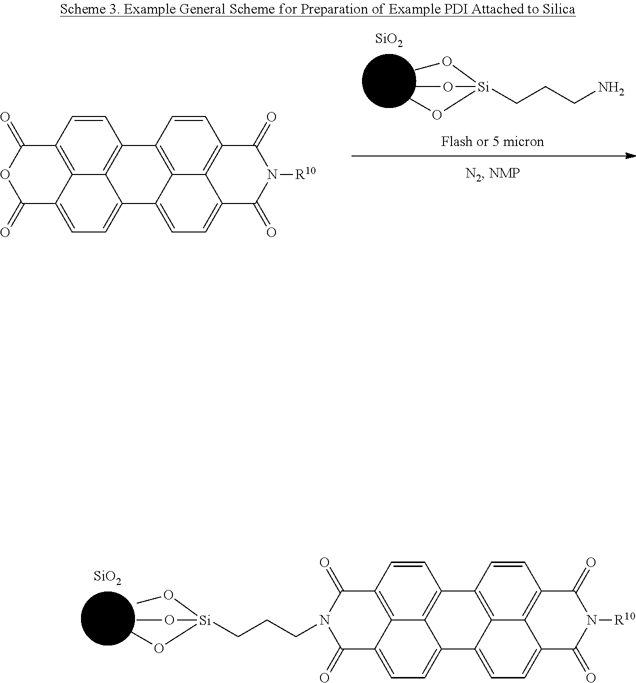

In the scheme above, —R¹⁰ generically includes -L¹-N(R¹)₃, where L¹, at each occurrence, is independently C₁₋₆alkylene or C₁₋₃alkylene-C═C—C₁₋₃alkylene; R¹, at each occurrence, is independently hydrogen, C₁₋₆alkyl, or C₁₋₆alkylene-N(R^{Y})ₙ, wherein n=1−3, and R^{Y}, at each occurrence, is independently hydrogen or C₁₋₄alkyl. The scheme above should not be construed as limiting in any way to the generalized description of the methods for covalently attaching the PDI-based fluorophores of the present disclosure to a solid support.

0.2 g (0.26 mmol) of FSG@APTMS was refluxed with 0.05 mmol PDI-1+ under N₂ in N-Methylpyrrolidone (NMP) for 3 h. After cooling at room temperature, the reaction mixture was filtered and washed with DMF (4×15 mL), followed by absolute ethanol (4×10 mL). The resultant reaction product FSG-PDI-1+ was dried under a vacuum oven at 60° C. for overnight.

0.2 g (0.26 mmol) of FSG@APTMS was also refluxed with 0.07 mmol PDI-3+ under N₂ in N-Methylpyrrolidone (NMP) for 3 h. After cooling at room temperature, the reaction mixture was filtered and washed with DMF (4×15 mL), followed by absolute ethanol (4×10 mL). The resultant reaction product FSG-PDI-3+ was dried under a vacuum oven at 60° C. overnight.

0.1 g (0.14 mmol) of 5 μSG@APTMS was refluxed with 0.05 mmol PDI-1+ under N₂ in N-Methylpyrrolidone (NMP) for 3 h. After cooling at room temperature, the reaction mixture was filtered and washed with DMF (4×15 mL), followed by absolute ethanol (4×10 mL). The resultant reaction product 5 μSG-PDI-1+ was dried under a vacuum oven at 60° C. overnight.

Finally, 0.1 g (0.14 mmol) of 5 μSG@APTMS was refluxed with 0.07 mmol PDI-3+ under N₂ in N-Methylpyrrolidone (NMP) for 3 h. After cooling at room temperature, the reaction mixture was filtered and washed with DMF (4×15 mL), followed by absolute ethanol (4×10 mL). The resultant reaction product 5 μSG-PDI-3+ was dried under a vacuum oven at 60° C. overnight.

Table 3 shows the structures of example PDI fluorophores covalently attached to silica.

TABLE 3
Structures of PDI fluorophores covalently attached to silica

| | |
|---|---|
| FSG-PDI- 1+: | 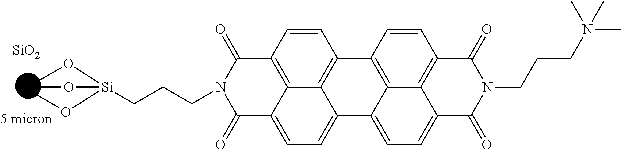 |
| 5μSG- PDI-1+: | 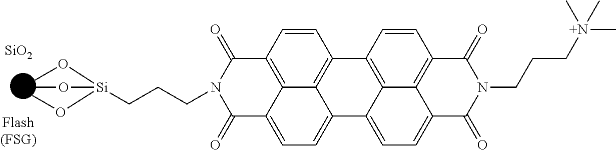 |
| 5μSG- PDI-3+ | 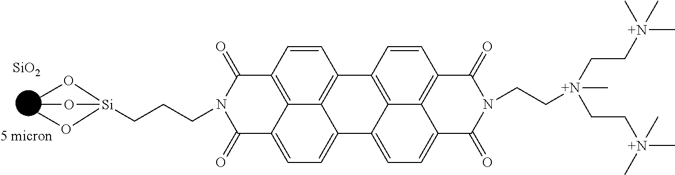 |
| FSG-PDI- 3+ | 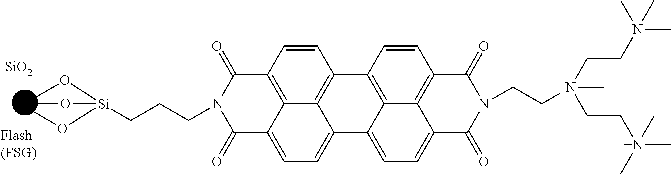 |

### Fluorescence Spectroscopic Studies

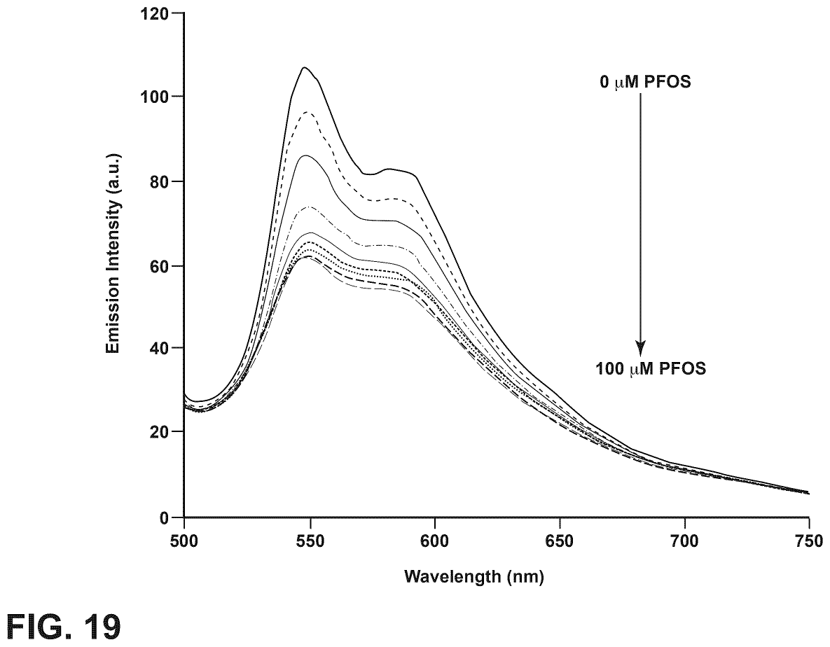

FIG. 19 shows a series of fluorescence emission spectra of 1 mg/mL 5 μSG-PDI-3+ in water in the presence of varying concentrations of PFOS (0-100 μM) at an excitation wavelength of 480 nm. With the addition of PFOS, the emission intensity of the 5 μSG-PDI-3+ gradually decreased and became saturated with PFOS in aqueous media, as the electrostatic interaction between PFOS and 5 μSG-PDI-3+ resulted in saturation. The decrease in emission intensity, particularly at 547 nm, illustrates the ability of PFOS to quench the emissions of 5 μSG-PDI-3+.

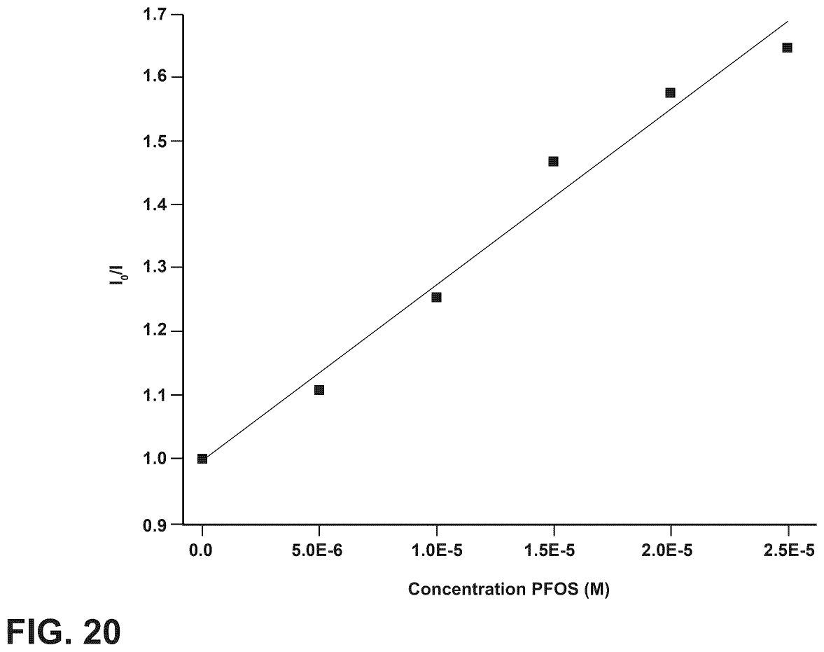

FIG. 20 shows interaction dynamics between 5 μSG-PDI-3+ and the quencher in a Stern-Volmer plot. The Iₒ/I for [1 mg/mL] of 5 μSG-PDI-3+ as measured at an emission wavelength of 547 nm (when excited at an absorbance wavelength of 480 nm) as a function of the concentration of PFOS (0-2.5×10⁻¹ M). Using the Stern-Volmer plot, the limits of detection (LOD) calculated for 5 μSG-PDI-1+ was 1.50 μM (0.81 ppm). The Stern-Volmer constant, K_{SV}, value was calculated to be 2.77×10⁴ M⁻¹.

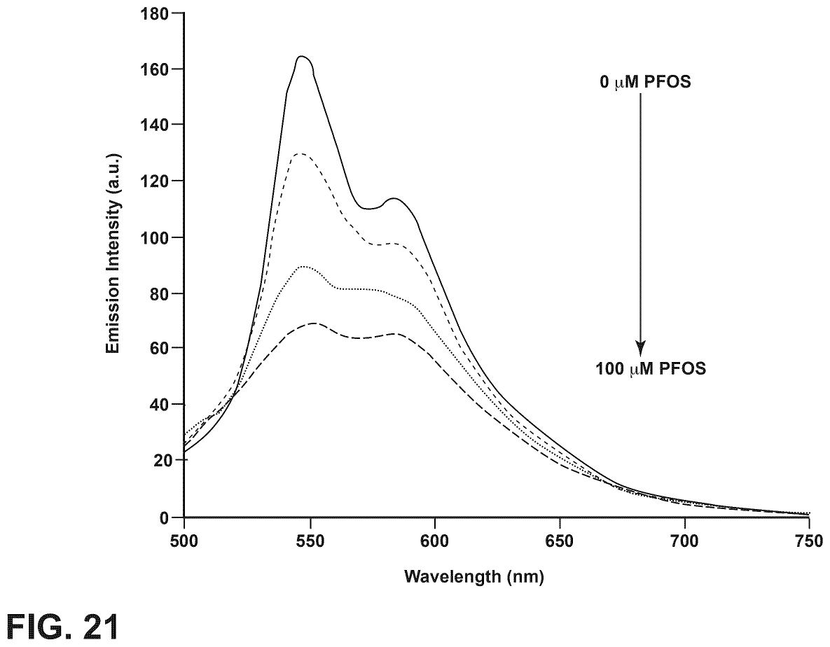

FIG. 21 shows a series of fluorescence emission spectra of 1 mg/mL 5 μSG-PDI-1+ in water in the presence of varying concentrations of PFOS (0-100 μM) at an excitation wavelength of 480 nm. With the addition of PFOS, the emission intensity of the 5 μSG-PDI-1+ gradually decreased and became saturated with PFOS in aqueous media, as the electrostatic interaction between PFOS and 5 μSG-PDI-1+ resulted in saturation. The decrease in emission intensity, particularly at 547 nm, illustrates the ability of PFOS to quench the emissions of 5 μSG-PDI-1+. Fluorescence peaks at 547 nm decreased as PFOS concentration increased.

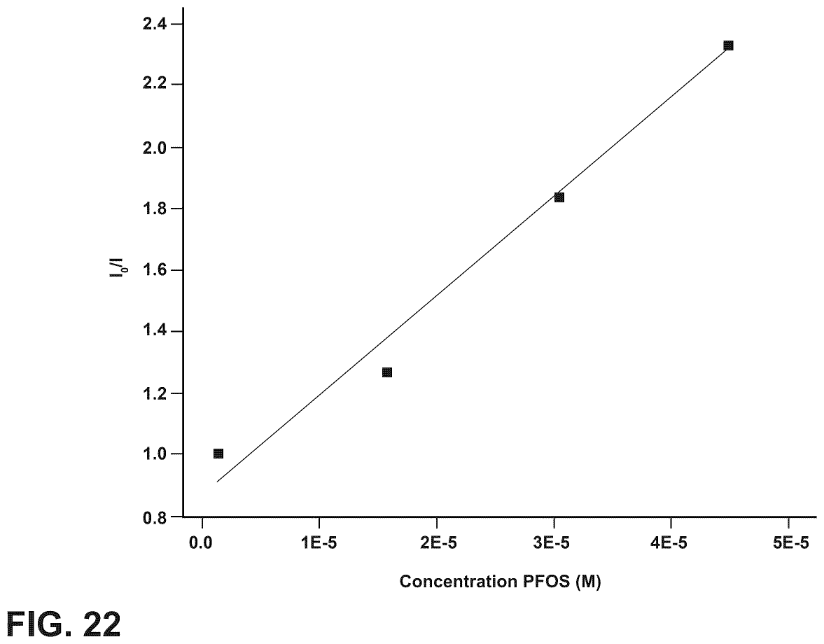

FIG. 22 shows interaction dynamics between 5 μSG-PDI-1+ and the quencher in a Stern-Volmer plot. The Iₒ/I for [1 mg/mL] of 5 μSG-PDI-1+ as measured at an emission wavelength of 547 nm (when excited at an absorbance wavelength of 480 nm) as a function of the concentration of PFOS (0-4.5×10⁻⁵ M). Using the Stern-Volmer plot, the limits of detection (LOD) calculated for 5 μSG-PDI-1+ was 1.50 μM (0.81 ppm). The Stern-Volmer constant, K_{SV}, value was calculated to be 3.1×10⁴ M⁻¹.

The method disclosed herein is further summarized in the following paragraphs and is further characterized by combinations of any and all various aspects described herein.

According to one aspect of the present disclosure, a method for detecting perfluorooctane sulfonic acid (PFOS) in an aqueous sample includes exciting the aqueous sample alone at a wavelength in a range of approximately 480 to 500 nm, contacting the aqueous sample with a detection reagent comprising a perylene-diimide-based cationic fluorophore, exciting the detection reagent and aqueous sample combination at a wavelength in a range of approximately 480 to 500 nm, and quantifying a change in a fluorescence intensity of the detection reagent while in contact with the sample relative to the fluorescence intensity of the detection reagent prior to being contacted with the sample.

According to yet another aspect of the present disclosure, the detection reagent and aqueous sample combination are excited at a wavelength in a range of approximately 480 to 490 nm.

According to yet another aspect of the present disclosure, the change in fluorescence intensity is quantified at an emission wavelength of 520-700 nm.

According to yet another aspect of the present disclosure, the change in fluorescence intensity is quantified at an emission wavelength of 540-600 nm.

According to yet another aspect of the present disclosure, a limit of detection for said method is about 1 to about 50 ppb.

According to another aspect of the present disclosure, a method for detecting perfluorooctane sulfonic acid (PFOS) in a sample, the method includes contacting the sample with a detection reagent including a compound of formula (I), or a salt thereof:

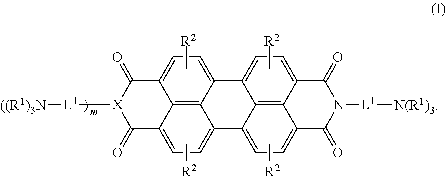

wherein:
X is either N or O, wherein when X is N, m is 1 and wherein when X is O, m is 0;
L¹, at each occurrence, is independently C₁₋₆alkylene or
C₁₋₃alkylene-C═C—C₁₋₃alkylene;
R¹, at each occurrence, is independently hydrogen, C₁₋₆alkyl, or
C₁₋₆alkylene-N(R^{Y})ₙ, wherein n=1−3.
R², at each occurrence, is independently hydrogen, C₁₋₆alkyl, halogen, or —O(R^{Y});
and
R^{Y}, at each occurrence, is independently hydrogen or C₁₋₄alkyl; and quantifying a change in a fluorescence intensity of the detection agent while in contact with the sample relative to the fluorescence intensity of the detection agent prior to being contacted with the sample.

According to another aspect of the present disclosure, R², at each occurrence, is independently hydrogen.

According to yet another aspect of the present disclosure, the compound of formula (I), or a salt thereof, is the compound of formula (Ia), or a salt thereof:

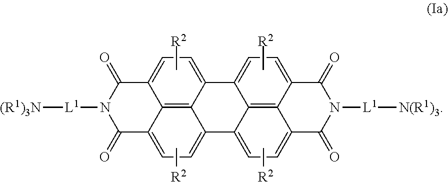

According to another aspect of the present disclosure, the compound of formula (I), or a salt thereof, is the compound of formula (Ib), or a salt thereof:

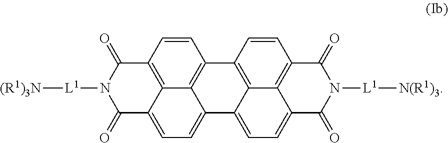

According to yet another aspect of the present disclosure, the compound of formula (I), or a salt thereof, is the compound of formula (Ic), or a salt thereof:

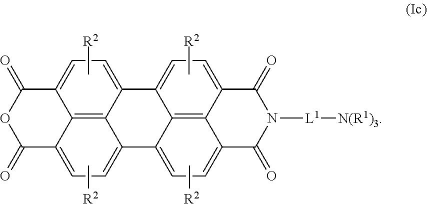

According to another aspect of the present disclosure, the compound of formula (I), or a salt thereof, is the compound of formula (Id), or a salt thereof:

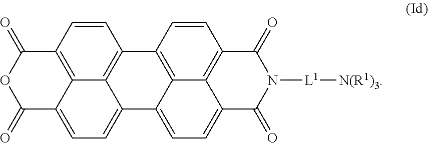

According to yet another aspect of the present disclosure, L¹ is C₁₋₆alkylene.

According to another aspect of the present disclosure, R¹ is C₁₋₆alkyl or C₁₋₆alkylene-N(R^{Y})ₙ.

According to yet another aspect of the present disclosure, n=3.

According to another aspect of the present disclosure, R^{Y} is C₁₋₄alkyl.

According to yet another aspect of the present disclosure, the compound of formula (I), or a salt thereof, is selected from the group consisting of:

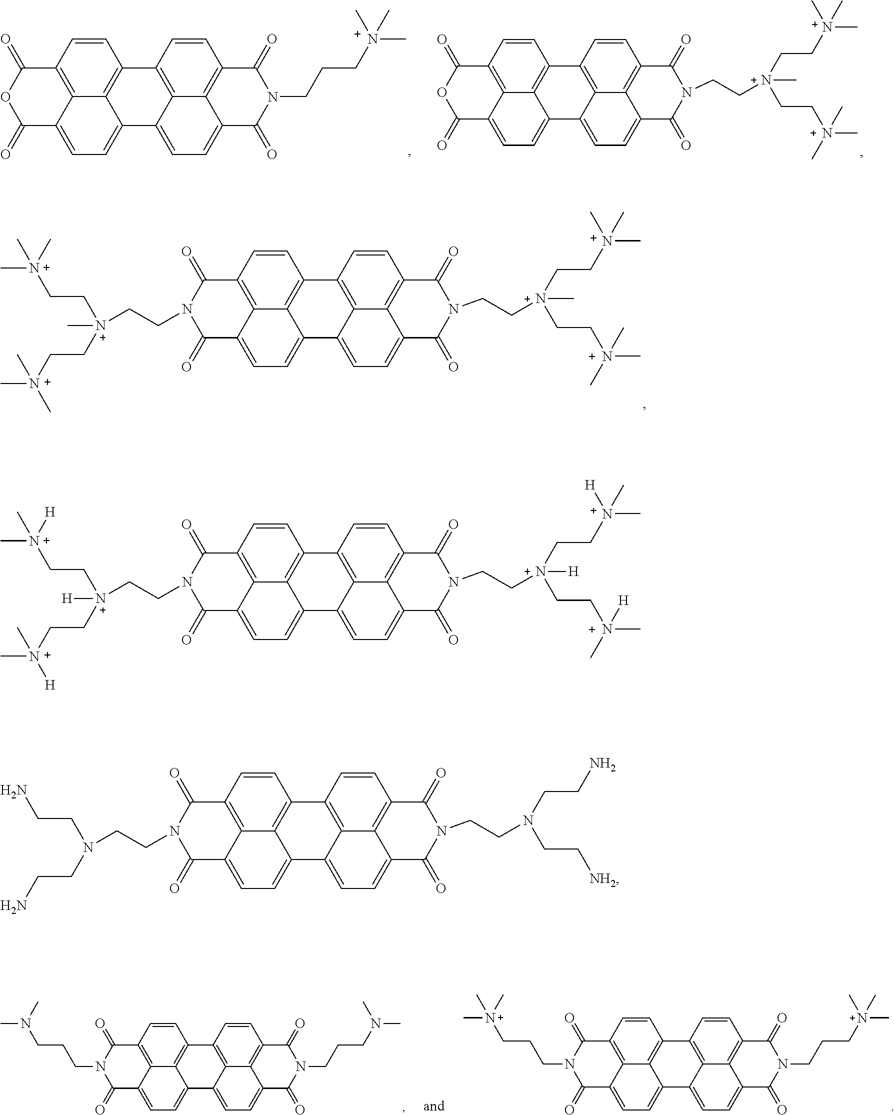

According to another aspect of the present disclosure, the compound of formula (I), or salt thereof, is noncovalently adhered to a surface.

According to yet another aspect of the present disclosure, the surface includes a cellulose, a silica, a polymer, a resin, or a combination thereof.

According to another aspect of the present disclosure, the compound of formula (I), or a salt thereof, is dissolved in a solvent.

According to yet another aspect of the present disclosure, the compound of formula (I), or a salt thereof, includes at least one counterion.

According to another aspect of the present disclosure, the counterion is selected from iodide, chloride, bromide, and combinations thereof.

According to yet another aspect of the present disclosure, the change in fluorescence intensity is decreased in fluorescence intensity.

According to another aspect of the present disclosure, the detection reagent is excited at an excitation wavelength of 480-500 nm.

According to yet another aspect of the present disclosure, the change in fluorescence intensity is quantified at an emission wavelength of 540-600 nm.

According to another aspect of the present disclosure, the detection reagent has a limit of detection of about 1 to about 50 ppb.

According to another aspect of the present disclosure, a method for detecting perfluorooctane sulfonic acid (PFOS) in a sample, includes contacting the sample with a detection reagent including a compound of formula (II), or a salt thereof:

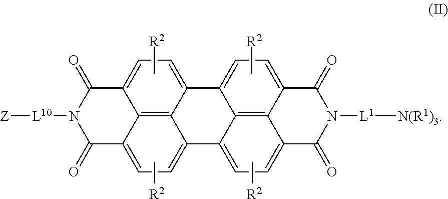

wherein:
L¹ is C₁₋₆alkylene or C₁₋₃alkylene-C═C—C₁₋₃alkylene;
R¹, at each occurrence, is independently hydrogen, C₁₋₆alkyl, or
C₁₋₆alkylene-N(R^{Y})ₙ, wherein n=1−3;
R², at each occurrence, is independently hydrogen, C₁₋₆alkyl, halogen, or —O(R^{Y});
   and
R^{Y}, at each occurrence, is independently hydrogen or C₁₋₄alkyl;
L¹⁰ is a linker;
Z is a solid support; and
   quantifying a change in a fluorescence intensity of the detection agent while in contact with the sample relative to the fluorescence intensity of the detection agent prior to being contacted with the sample.

According to another aspect of the present disclosure, R², at each occurrence, is independently hydrogen.

According to yet another aspect of the present disclosure, L¹ is C₁₋₆alkylene.

According to another aspect of the present disclosure, R¹ is C₁₋₆alkyl or C₁₋₆alkylene-N(R^{Y})ₙ.

According to yet another aspect of the present disclosure, n=3.

According to another aspect of the present disclosure, R^{Y} is C₁₋₄alkyl.

According to yet another aspect of the present disclosure, L¹⁰ is C₃alkylene.

According to another aspect of the present disclosure, the solid support Z is formed of a material including a silica, a polymer and a resin, and combinations thereof.

According to yet another aspect of the present disclosure, the compound of formula (II), or a salt thereof, is the reaction product of an amine-functionalized solid support and the compound of formula (Ic):

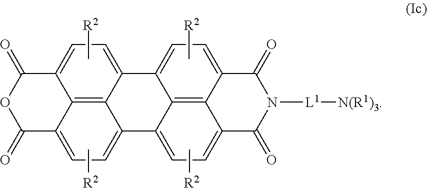

According to another aspect of the present disclosure, the amine-functionalized solid support includes —C₁₋₁₀alkylene-NH₂ chemically coupled to the solid support, and wherein L¹⁰ includes C₁₋₁₀alkylene.

According to yet another aspect of the present disclosure, the amine-functionalized solid support is amine-functionalized silica.

According to another aspect of the present disclosure, the amine-functionalized silica is a product of a reaction between silica and either APTMS or APTES, and wherein L¹⁰ includes C₃alkylene.

According to yet another aspect of the present disclosure, the compound of formula (II), or a salt thereof, includes at least one counterion.

According to another aspect of the present disclosure, the counterion is selected from iodide, chloride, bromide, and combinations thereof.

According to yet another aspect of the present disclosure, the change in fluorescence intensity is a decrease in fluorescence intensity.

According to another aspect of the present disclosure, the detection reagent is excited at an excitation wavelength of 480-500 nm.

According to yet another aspect of the present disclosure, the change in fluorescence intensity is quantified at an emission wavelength of 520-700 nm.

According to another aspect of the present disclosure, the detection reagent has a limit of detection of about 1 to about 50 ppb.

It will be apparent to those of ordinary skill in the relevant art that suitable modifications and adaptations to the methods, processes, and applications described herein can be made without departing from the scope of any embodiments or aspects thereof. The methods provided are exemplary and are not intended to limit the scope of any of the specified embodiments. All of the various embodiments, aspects, and options disclosed herein can be combined in any variations or iterations. The scope of the methods and processes described herein include all actual or potential combinations of embodiments, aspects, options, examples, and preferences herein described. The exemplary methods described herein may substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present disclosure, and further it is to be understood that such concepts are intended to be covered by the following claims unless these claims by their language expressly state otherwise.

What is claimed is:

1. A method for detecting perfluorooctane sulfonic acid (PFOS) in a sample, the method comprising:
contacting the sample with a detection reagent comprising a compound of formula (I), or a salt thereof: 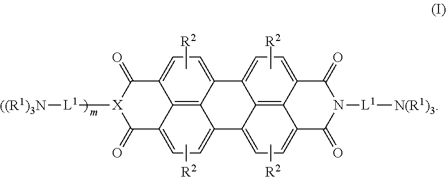
wherein:
X is either N or O, wherein when X is N, m is 1 and wherein when X is O, m is 0;
L¹, at each occurrence, is independently C₁₋₆alkylene or
C₁₋₃alkylene-C═C—C₁₋₃alkylene;
R¹, at each occurrence, is independently hydrogen, C₁₋₆alkyl, or
C₁₋₆alkylene-N(R^{y})ₙ, wherein n=1−3;
R², at each occurrence, is independently hydrogen, C₁₋₆alkyl, halogen, or —O(R^{y}); and
R^{y}, at each occurrence, is independently hydrogen or C₁₋₄alkyl; and
quantifying a change in a fluorescence intensity of the detection agent while in contact with the sample relative to the fluorescence intensity of the detection agent prior to being contacted with the sample.

2. The method for detecting perfluorooctane sulfonic acid (PFOS) in a sample of claim 1, wherein R², at each occurrence, is independently hydrogen.

3. The method for detecting perfluorooctane sulfonic acid (PFOS) in a sample of claim 1, wherein the compound of formula (I), or a salt thereof, is the compound of formula (Ia), or a salt thereof:

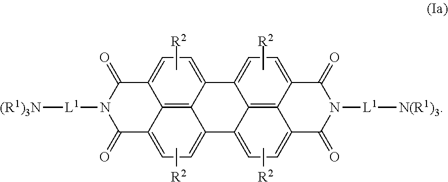

4. The method for detecting perfluorooctane sulfonic acid (PFOS) in a sample of claim 3, wherein the compound of formula (I), or a salt thereof, is the compound of formula (Ib), or a salt thereof:

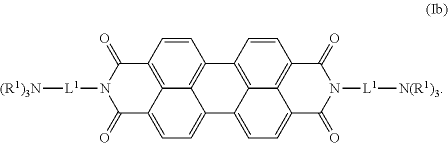

5. The method for detecting perfluorooctane sulfonic acid (PFOS) in a sample of claim 1, wherein the compound of formula (I), or a salt thereof, is the compound of formula (Ic), or a salt thereof:

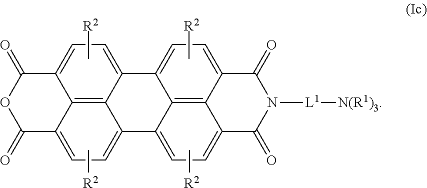

6. The method for detecting perfluorooctane sulfonic acid (PFOS) in a sample of claim 5, wherein the compound of formula (I), or a salt thereof, is the compound of formula (Id), or a salt thereof:

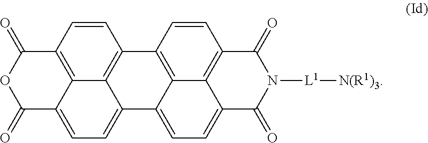

7. The method for detecting perfluorooctane sulfonic acid (PFOS) in a sample of claim 6, wherein L¹ is C₁₋₆alkylene.

8. The method for detecting perfluorooctane sulfonic acid (PFOS) in a sample of claim 7, wherein R¹ is C₁₋₆alkyl or C₁₋₆alkylene-N(R^{x})ₙ.

9. The method for detecting perfluorooctane sulfonic acid (PFOS) in a sample of claim 8, wherein n=3.

10. The method for detecting perfluorooctane sulfonic acid (PFOS) in a sample of claim 9, wherein R^{y} is C₁₋₄alkyl.

11. The method for detecting perfluorooctane sulfonic acid (PFOS) in a sample of claim 2, wherein the compound of formula (I), or a salt thereof, is selected from:

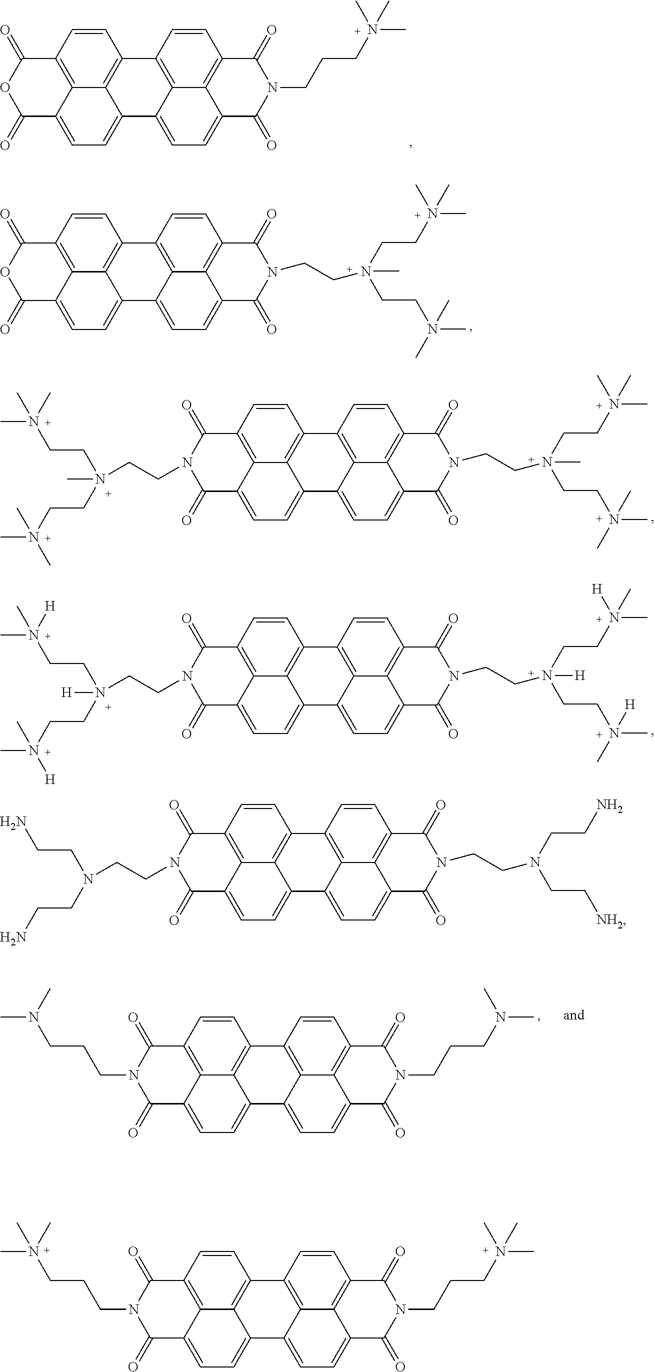

12. The method for detecting perfluorooctane sulfonic acid (PFOS) in a sample of claim 1, wherein the compound of formula (I), or salt thereof, is noncovalently adhered to a surface, wherein the surface includes at least one of cellulose, silica, polymer, resin, or a combination thereof.

13. The method for detecting perfluorooctane sulfonic acid (PFOS) in a sample of claim 1, wherein the compound of formula (I), or a salt thereof, includes at least one counterion selected from iodide, chloride, bromide, and combinations thereof.

14. A method for detecting perfluorooctane sulfonic acid (PFOS) in a sample, the method comprising:
contacting the sample with a detection reagent comprising a compound of formula (II), or a salt thereof: 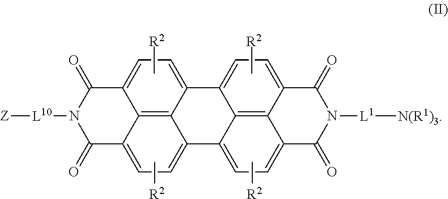
wherein:
L¹ is C₁₋₆alkylene or C₁₋₃alkylene-C═C—C₁₋₃alkylene;
R¹, at each occurrence, is independently hydrogen, C₁₋₆alkyl, or
C₁₋₆alkylene-N(R^{x})ₙ, wherein n=1−3;
R^{y}, at each occurrence, is independently hydrogen, C₁₋₆alkyl, halogen, or —O(R^{y}); and
R^{X}, at each occurrence, is independently hydrogen or C₁₋₄alkyl;
L¹⁰ is a linker;
Z is a solid support; and
quantifying a change in a fluorescence intensity of the detection agent while in contact with the sample relative to the fluorescence intensity of the detection agent prior to being contacted with the sample.

15. The method for detecting perfluorooctane sulfonic acid (PFOS) in a sample of claim 14, wherein the solid support Z is formed of a material selected from silica, polymer, resin, and combinations thereof.

16. The method for detecting perfluorooctane sulfonic acid (PFOS) in a sample of claim 14, wherein the compound of formula (II), or a salt thereof, is the reaction product of an amine-functionalized solid support and the compound of formula (Ic):

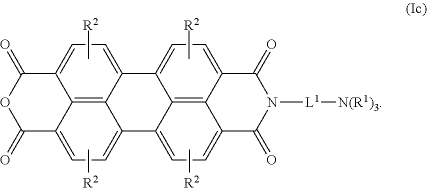

17. The method for detecting perfluorooctane sulfonic acid (PFOS) in a sample of claim 16, wherein the amine-functionalized solid support is amine-functionalized silica.

18. A method for detecting perfluorooctane sulfonic acid (PFOS) in an aqueous sample, the method comprising:
exciting the aqueous sample alone at a wavelength in a range of approximately 480 to 500 nm;
contacting the aqueous sample with a detection reagent comprising a perylene-diimide-based cationic fluorophore;
exciting the detection reagent and aqueous sample combination at a wavelength in a range of approximately 480 to 500 nm; and
quantifying a change in a fluorescence intensity of the detection reagent while in contact with the sample relative to the fluorescence intensity of the detection reagent prior to being contacted with the sample.

19. A method for detecting perfluorooctane sulfonic acid (PFOS) in an aqueous sample of claim 18, wherein the change in fluorescence intensity is quantified at an emission wavelength of 520-700 nm.

20. A method for detecting perfluorooctane sulfonic acid (PFOS) in an aqueous sample of claim 18, wherein a limit of detection for said method is about 1 to about 50 ppb.
